(19) United States
(12) Patent Application Publication
(10) Pub. No.: US 2007/0033871 A1
(43) Pub. Date: Feb. 15, 2007

(54) Partially contained, reduced weight plant growth medium

(76) Inventors: Michael Kelly, Blaine, MN (US); Jay Hudson, Coon Rapids, MN (US)

Correspondence Address: MOORE & HANSEN, PLLP, 225 SOUTH SIXTH ST, MINNEAPOLIS, MN, 55402, US

(21) Appl. No.: 11/482,332

(22) Filed: Jul. 7, 2006

Related U.S. Application Data
(60) Provisional application No. 60/697,825, filed on Jul. 7, 2005.

(51) Int. Cl. A01H 3/00

(52) U.S. Cl. 47/58.1 R

(57) ABSTRACT

An at least partially contained, reduced weight (PCRW) plant growth medium, providing a sand/soil matrix including a sand/soil mixture in which plants can grow. The sand/soil matrix preferably includes sand/soil displacement structures proximate a plurality of isolated locations where the uninterrupted sand/soil depth in that zone is less than in a first set of isolated locations. The sand/soil displacement structures having a weight per unit area that is less than the weight per unit area of the sand/soil mixture, thereby reducing the weight of the growth medium, while still providing a suitable sand/soil profile for plant growth. A building structure, a spectator pavilion and a modular plant growth system including such a PCRW plant growth medium, or a plurality thereof, are also disclosed as are methods of building a reduced weight green space on a roof of a building structure.

### RELATED APPLICATIONS

The present application claims priority to and the benefit of co-pending U.S. Patent Application Ser. No. 60/697,825 filed Jul. 7, 2005, entitled PARTIALLY CONTAINED, REDUCED WEIGHT PLANT GROWTH MEDIUM, the enclosure of which is incorporated herein by reference in its entirety.

### FIELD OF THE INVENTION

The present invention relates to at least partially contained, reduced weight plant growth mediums, preferably for residence upon a roof of a building structures or for incorporation into a larger growth medium such as a modular playing field or green space including a plurality of field or growth medium units, which can be assembled together to form a larger field area. The soil profile of the reduced weight plant growth medium preferably includes displacement structures that effectively displace a portion of the sand/soil mixture in the sand/soil profile to reduce the weight of the sand/soil profile by substituting air for the sand/soil mixture within the sand/soil profile. A building structure, a spectator pavilion and a modular plant growth system, each of which preferably include a plurality of at least partially contained, reduced weight plant growth medium units, are also disclosed. A spectator pavilion including a single such growth medium is also disclosed. In preferred embodiments of the invention, a subsurface fluid distribution system or a subsurface irrigation and drainage system, otherwise referred to in the marketplace as an Evaporative Control System (or, as referenced commercially, “an ECS system”) is also provided. In yet other embodiments of the present invention, the sand/soil mixture in an upper part of the sand/soil profile will include load-bearing enhancers, preferably polymeric mesh elements to enhance the load-bearing characteristics of the sand/soil profile.

### BACKGROUND

It is becoming increasingly important for urban dwellings and offices to have green spaces that absorb water and improve the environment. Green space creates oxygen, clean air, clean water, absorbs and thereby reduces heat and is esthetically pleasing. Storm-water surge is reduced because of the extra time that it takes water to get through the vegetation and soil profile, before it reaches the storm-water system.

It will be appreciated that in urban areas, generally filled with non-porous asphalt or cement surfaces, green space comes at a premium. Parks and other open space that may provide some greenery in urban areas are generally limited in number and generally come under development pressure. Although this pressure is generally balanced by the public's desire for the broad enjoyment that parks and other green space can provide, it is almost always the case that new green space of this type cannot be created without extremely strong public pressure, which is virtually never strong enough to over come the financial limitations generally associated with such a project.

Areas, such as roof-tops, parking garage plazas and the like are generally available, however, for utilization to provide such green space and the accompanying environmental enhancements that attach to such space in urban areas. These facilities are generally sensitive to weight loads, however. This is especially true in the case of previously constructed commercial buildings whose architects did not anticipate a roof-top green space. Furthermore, in new construction, it will be appreciated that an architect would need to plan for the addition of a green space on the top of a roof because of the additional weight of the soil needed to grow grass or other ground covers. If such a green space is extremely heavy, it will be further appreciated that further construction expense will be required to provide the needed structural support for the added weight.

Plants generally require a certain soil depth or depth of profile to facilitate correct moisture and air levels in plant root-zones. Larger plants, such as bushes and trees, may also need greater root-zone depth to anchor the plants. In order to provide the appropriate amount of soil depth for planting on a roof or other structure, substantial expenditures may be required to provide the needed structural support to permit such a soil depth or soil profile.

A suitable soil depth or soil profile is generally required to provide a positive root-zone that will enhance plant growth. The most positive root-zone is generally made up of about 50% solids or particle, about 25% water and about 25% air. When using clean washed sand as a particle matrix, the size and shape of the sand will determine the water holding capacity of the profile. The smaller the particles, the more water will be held between them. The larger the particles, the greater the drainage and the higher the potential for more air space.

It is generally believed that the depth of the soil profile has to be deep enough that it allows for a positive root-zone with the correct amount of air and water in order to have healthy plant growth. If the profile is too short, it will saturate easily and not provide enough air for suitable growth. If the profile is too deep, the water will drain to the bottom of the profile and not be available to the roots of the plants for the plant's growth.

Subsurface irrigation systems have been developed to enhance rates of water flow through subsurface soil profiles and to maximize the efficiency of the use of water that is available. One such system is disclosed in U.S. Pat. No. 5,921,711 to Jones Sipaila. The subsurface fluid distribution system disclosed by the Sipaila patent has been successfully sold in the marketplace as an Evaporative Control System or “ECS system.”

The ECS System provides a constant reservoir of water in the bottom of a tray having an impermeable membrane. This system allows water to wick upward instead of requiring surface watering. This system allows one to build a soil profile height by the characteristics of the growing medium. If sand is used, it is very predictable. If other growth media are used, uniformity is important so that the degree of capillary rise can be predicted and/or measured.

Since green-roof tops and athletic field modules are weight sensitive, architects and builders must make allowances to accommodate the weight of each of the profiles as well as provide the required depth of profile to provide optimum growing conditions.

Modern athletic playing fields are occasionally constructed to include a plurality of modules, each of which might consist of a self contained soil profile providing a medium to support plant growth, preferably natural grass. These modules can be moveable, e.g. the playing surface at Reliant Stadium in Houston, Tex. Each soil profile module may be quite heavy, however, so moving each module is a challenge that requires some consideration or allowance for the weight created by the soil profile.

A somewhat newer concept is to provide a single transportable playing field that is essentially a single, but very large, module that maybe moved on rails, wheels or the like; e.g. the new Cardinal's Stadium being constructed in Glendale, Ariz.

Transporting the 12 million pound playing field, however, it is undoubtedly a challenge.

It will be appreciated from the foregoing, therefore, that prior art plant growth mediums and methods of providing such plant growth mediums are limited in what they provide and in the types of situations in which they can be provided. It also will be appreciated that further enhancements of the technology associated with providing “green space” and the provision of plant growth mediums are needed. The present invention provides solutions for these and other challenges.

### SUMMARY

The present invention includes an at least partially contained, reduced weight plant growth medium (PCRW plant growth medium). The at least partially contained, reduced weight plant growth medium preferably includes a sand/soil matrix in which plants can grow. The sand/soil matrix includes a sand/soil mixture having a first weight per unit of area. In preferred embodiments, the sand/soil matrix is constructed and arranged to provide a first uninterrupted sand/soil depth of the sand/soil mixture in a plurality of first zones proximate a first set of isolated locations that is sufficient to support growth of plant species and a second sand/soil depth of the sand/soil mixture in a plurality of second zones proximate a second set of isolated locations that is less than the first uninterrupted sand/soil depth at the plurality of first zones, wherein the sand/soil matrix includes a displacement structure, preferably an air incorporating sand/soil displacement structure, preferably an air incorporating sand/soil displacement structure selected from the group consisting of (a) an interconnected structure constructed and arranged to (1) incorporate an air containing void into the sand/soil matrix and (2) prevent the sand/soil mixture from filling the air containing void in at least one of the plurality of second zones proximate the second set of isolated locations and (b) an air incorporating sand/soil displacement structure that effectively displaces a portion of the sand/soil mixture in the sand/soil matrix in at least one of the plurality of second zones proximate the second set of isolated locations, the air incorporating sand/soil displacement structure incorporating sufficient air into the at least one of the plurality of second zones such that at least one of the plurality of second zones will have a second weight per unit area, that is substantially less than the first weight per unit area. The displacement structure will preferably be made of a material that is either lighter than sand or a material that either encapsulates or captures sufficient air or creates a pocket for or sufficient shelter for a pocket of air, so as to sufficiently displace an amount of the sand/soil mixture to make the sand/soil matrix proximate a second zone weigh less than the sand/soil mixture. In alternate embodiments the displacement structure may also be constructed of polymeric materials, wood or ceramic materials that are either lighter than sand, e.g. rubber, nylon, polyethylene and the like, most woods and clay ceramics and the like, or such materials that are used to incorporate other materials that are even lighter, so as to have the effect of creating a displacement structure that can be used to displace the sand/soil mixture, thereby reducing the weight per unit area of the sand/soil matrix proximate the displacement structure. The sand/soil matrix is preferably constructed and arranged to provide an uninterrupted sand/soil depth of at least about 4 inches, preferably about 7 inches, more preferably about 9 inches, even more preferably about 10 inches and most preferably about 12 inches of the sand/soil mixture in a plurality of first zones proximate a first set of isolated locations; an uninterrupted sand/soil depth of at least about 1 inch, preferably about 3 inches, more preferably about 4 inches, even more preferably about 5 inches and most preferably about 6 inches or more of the sand/soil mixture in a plurality of second zones proximate a second set of isolated locations. The sand/soil matrix preferably includes a displacement structure that effectively displaces a portion of the sand/soil mixture preferably proximate each of the second set of isolated locations. The air incorporating sand/soil displacement structure will preferably be constructed and arranged to displace a portion of the sand/soil mixture proximate each of the second set of isolated locations. It will be understood that the term “sand/soil” as used throughout the disclosure refers not only to sand and soil but to and particulate material that can support plant growth (i.e. compost, zeolite, remiculite, pearlite, expanded shale, organic fiber, silica particles, lava rock particles, crum rubber particles, polypropylene particles, Styrofoam particles and the like). Sand is preferred because it does not compress and compact over time due to decomposition. With respect to the weight of the sand/soil mixture, it will be appreciated that moisture will also be included in the mixture when the sand/soil mixture is in a plant growth medium and that the sand/soil displacement structures will generally displace moisture as well as the sand/soil mixture in the locations proximate the second zones, further diminishing the weight of the respective plant growth mediums including these displacement structures, while still providing sufficient sand/soil depth or depth of profile to allow hearty plant growth in such a plant growth medium.

The present invention provides a plant growth system having a reduced weight growing media, without diminishing the height of the plant growth media profile (growing profile) or the capillary activity of the growth media. Its function and properties preferably include a system that: a) allows raising the height of the growing profile without adding significant weight to the growth media; b) has generally sand/soil mixture displacement members; c) can preferably include sand, sand/soil, sand/peat, sand/compost, sand/vermeculite, sand/perilite, various potting soils and the like; and d) preferably includes sand/soil mixture displacement members spaced so that continuous capillary rise or drainage will continue to occur. In constructing a PCRW growth medium, it is believed to be important to have a growing profile having the right height to provide the right mixture of air and water in the soil for good root development and growth. The sand/soil mixture displacement members are preferably made of foam, empty water bottles, air filled pipes, air float rafts with capillary passage ways, and the like. The sand/soil displacement structures preferably have enough weight placed on top of them within respective preferred PCRW plant growth mediums to prevent or minimize the degree to which they rise with respect to the sand/soil mixture when exposed to climatic heating, cooling (e.g. “frost-heaving”) and/or excessive stormwater exposure or in alternate embodiments (not shown) they can be secured to another structure, e.g. the tray structure.

In preferred embodiments, the air incorporating sand/soil displacement structures are selected from the group consisting of (a) an interconnected structure constructed and arranged to (1) incorporate an air containing void into the sand/soil matrix and (2) prevent the sand/soil mixture from filling the air containing void in at least one of the plurality of second zones proximate the second set of isolated locations and (b) an air incorporating sand/soil displacement structure that effectively displaces a portion of the sand/soil mixture in the sand/soil matrix in at least one of the plurality of second zones proximate the second set of isolated locations, the displacement structure incorporating sufficient air into the at least one of the plurality of second zones such that at least one of the plurality of second zones will have a second weight per unit area, that is substantially less than the first weight per unit area.

In preferred embodiments the air incorporating sand/soil displacement structure is a solid structure made of an air encapsulating foam material having a predetermined height and a plurality of openings; each opening preferably running an entire length of the predetermined height to permit the sand/soil mixture to occupy the entire predetermined height in each opening, thereby providing an uninterrupted sand/soil depth proximate each of the plurality of openings of at least about two inches more than the length of the predetermined height and preferably an uninterrupted sand/soil depth in the upper root-zone sufficient to hold the sand/soil displacement structure(s) in place within the sand/soil matrix.

In further embodiments of the present invention, the sand/soil matrix will preferably include a plurality of air incorporating sand/soil displacement structures effective to displace a portion of the sand/soil mixture proximate each of the second set of isolated locations. The preferred air incorporating sand/soil displacement structures will be selected from the group consisting of (a) air encapsulating foam material; (b) an enclosed air containing compartment and a concave air impermeable structure that is constructed; and (c) arranged to capture and retain air when the air impermeable structure is inverted and open to the bottom. In other embodiments, the sand/soil matrix will include a plurality of air and water permeable structures constructed and arranged to (1) incorporate an air containing void into the sand/soil matrix and (2) prevent the sand/soil mixture from filling the air containing void in at least one of the plurality of second zones proximate the second set of isolated locations effective to displace a portion of the sand/soil mixture proximate each of the second set of isolated locations.

In preferred embodiments of the present invention, the at least partially contained, reduced weight plant growth medium will include a tray structure in which the sand/soil matrix resides; the tray structure having a bottom and four sides such that water can be contained in at least a lower portion of the tray structure. Preferably, this embodiment of the present invention will include a subsurface fluid distribution chamber. The subsurface fluid distribution chamber will preferably include an enclosure having a fluid transfer opening. At least a portion of the enclosure will preferably be a two-walled structure including an outer shell having a plurality of apertures formed along a first lateral portion thereof and an inner shell attached to the outer shell in a spaced apart manner of the type disclosed in the Sipaila patent referenced above. The inner shell 60 will preferably have a plurality of inner shell apertures 62 formed along a first lateral portion thereof, wherein the inner shell apertures 62 define, relative to the enclosure, a subsurface fluid level that is greater than a fluid level defined by the outer shell apertures 64; and wherein each of the plurality of inner shell apertures 62 is of a size enabling the formation of an infiltrated particle bed or saturation zone 68 adjacent to the outer shell apertures 64. The saturation zone 68 will preferably be at a level substantially equal to the fluid level defined by the inner shell apertures 62.

In alternate embodiments of the present invention, the at least partially contained, reduced weight plant growth medium (PCRW plant growth medium) can provide a natural drainage system, a subsurface fluid distribution system or ECS system of the type described immediately above in the prior paragraph, or a perched water table system. The ECS system will have an evaporative control system chamber and the sand/soil matrix will extend into a saturation zone in which free water is retained within the tray. In a natural drainage system, the at least partially contained, reduced weight plant growth medium will include a tray structure in which the sand/soil matrix resides, just as it does in an ECS system, however, the natural drainage system will not include an evaporative control system chamber to transfer fluids and retained water at the bottom of the tray will flow out of the tray at the level of any fluid transfer pipes or “weep holes” that allow free water to exit the alternate tray structure. In a perched water table system, the sand/soil matrix is supported, either by a stone drainage layer, or alternately by a tight mesh material, either of which prevents the sand/soil matrix from continuing to migrate with any free water that may flow through the sand/soil matrix. In a perched water table system, the sand/soil matrix does not come into contact with a saturation zone where free water is retained within the system. Instead, free water flowing through the medium continues to flow through the sand/soil matrix until it leaves the matrix and the water remaining in the sand/soil matrix is water adhering to the surfaces of the particulate material in the sand/soil matrix and essentially no further free water remains.

Because the sand/soil matrix in the perched water table system is separated from free water when the water and the matrix reaches equilibrium, the perched water table system is more dependent upon receiving water from the top in order to maintain suitable plant growth and to provide suitable water to the roots in the upper root zone of the sand/soil matrix. Each of the systems described above have their applications and, in each case, it will be desirable in certain situations to reduce the weight of the PCRW plant growth medium by including displacement structures that displace a portion of the sand/soil mixture in the second set of locations within the sand/soil matrix in a manner that reduces the weight per unit area of the respective PCRW plant growth medium.

In preferred embodiments, a building structure is provided; the building structure including a structural support system; a roof connected with and constructed and arranged to be above at least a portion of the structural support system; and one at least contained, reduced weight plant growth medium of the present invention, preferably a plurality of the same, residing upon the roof.

In preferred embodiments, a spectator pavilion is provided including a spectator seating area including a plurality of seats and a playing area including a field area; the field area including a plurality of field units assembled together adjacent to one another to form the field area. Each field unit will preferably include an at least partially contained, reduced weight plant growth medium of the present invention.

In other preferred embodiments, the at least partially contained, reduced weight plant growth medium will be provided for residence upon a roof of a building structure. Still other preferred embodiments provide a modular plant growth system including a plurality of at least partially contained, reduced weight plant growth mediums of the present invention constructed and arranged in proximity to one another to provide an extended green space or field.

In preferred embodiments, the at least partially contained, plant growth mediums will be an ECS system, however, in alternate embodiments a perched water table system or a natural drainage system may be provided. A perched water table system, similar to that used at Reliant Stadium, in Houston, Tex., which has a 5 inch soil profile, may be provided, however, it would be preferable to have a greater soil profile, preferably more than about 4 inches, preferably equal to or greater than about 7 inches, even more preferably equal to or greater than more than about 9 inches, even more preferably equal to or greater than from about 10 inches to about 14 inches or more. Unlike an ECS system, however the sand/soil matrix rests upon an air and water permeable barrier, such as a polymeric mesh material, that will retain the sand/soil matrix, but allow air and water to pass through.

The present invention will also include a method of building an at least partially contained, reduced weight plant growth medium for residence upon a roof of a building structure and also for incorporation into a field area of a spectator pavilion or the like. It will be appreciated that the at least partially contained, reduced weight plant growth medium of the present invention may be provided as a component of any architecturally devised building project in which it is desirable to reduce the weight of a plant growth medium to meet restricted weight specifications.

It is an object of the present invention to provide a reduced weight plant growth medium for placement on or incorporation into a building structure so that the structural requirements for supporting the weight of the plant growth medium can be reduced in order to reduce the expenses associated with providing the structural for the incorporation of the plant growth medium.

It is a further object of the present invention to provide a building structure having an at least partially contained, reduced weight plant growth medium on a roof of the structure to provide the special amenities that attend to providing green space on the roof of a building structure, whether in an urban area or otherwise.

It is a further object of the present invention to provide an, at least partially contained, reduced weight plant growth medium that may be somewhat more easily transported from one place to another and subsequently assembled into a playing field that includes a plurality of such, at least partially contained, reduced weight growth mediums.

A soil profile or sand/soil matrix of the embodiments of the present PCRW plant growth mediums generally provides an upper root-zone, a lower root-zone and a drainage zone or layer. It will be appreciated that the incorporation of spacers into the soil profile will lighten the load but not compromise the total height of the profile needed. This is because total height of profile needed can be provided in a series of separated locations without significantly diminishing the access to water in the subsurface by roots in the upper root-zone. It is critical to receive adequate drainage when top surface watering is anticipated or access to water through capillary rise that is generally provided in an ECS system or perhaps a natural drainage system. If the profile is too shallow, a little water goes a long way. A 3″ profile of sand/soil mixture is saturated easily because the water generally won't gravitationally drain away. The capillary force attraction of the sand is greater than the gravitational pull downward, resulting in a profile with too high a concentration of moisture and too little air, each of which will diminish growth. The sand/soil profiles have to be built deep enough to allow the appropriate air-to-water ratio in the upper root-zone. Linking the upper root-zone to the drainage zone in an ECS system or a natural drainage system will do this adequately, so long as there is sufficient depth to the sand/soil matrix to allow free water to drain out of the upper root-zone.

The use of an air incorporating sand/soil displacement structure such as a light-weight closed cell spacer like Styrofoam or Geo-foam, air-filled bottles, compartmentalized air pockets, concave structures and arranged to exclude or displace the sand/soil mixture in the sand/soil matrix or the like will allow the user to obtain the height of the profile without the greater weight of a prior art growth medium having a sand/soil profile that may be as deep as 13″ or more without any zones where the sand/soil mixture is displaced to reduce the weight of the medium.

It is preferred that the system will allow the upper root-zone to connect with the drainage layer and preferably will be constructed and arranged to include columns of the sand/soil mixture extending from the top surface of the upper root-zone to the bottom of the lower root-zone at the bottom of the sand/soil matrix. The columns will act as wicks for vertical movement of water up and down the sand/soil profile. It will be appreciated that the drainage layer may also be called a drainage and water dispersing layer.

The addition of an ECS Chamber in an ECS System allows for a constant supply of water and more control over the drying and wetting cycles if an appropriate amount of water is maintained in a saturation zone in the bottom of the ECS tray.

If one uses a washed cement sand or mason sand as the sand/soil mixture, the optimum height of the profile will preferably be from about 5″ to about 18″, more preferably from about 6″ to about 17″, more preferably from about 8″ to about 15″ and most preferably from about 10″ to about 12″. In preferred embodiments, the user will have a semi-dry surface with a pore-space occupancy in a range of from about 5% water to about 25% water and about 75% air to about 95% air, which is generally considered to be relatively optimal. As one goes lower in the sand/soil profile, the water/air ratio will preferably balance at about 50% water and about 50% air in the upper-root zone. As one goes even lower, the percentage of water will increase until it becomes nearly 100% water near the bottom of the lower root-zone and the water completely fills the pore-space in a saturation zone that may extend up into a lower portion of the lower root-zone. The ECS System saves water because water is fed to the system below the surface or rainwater is absorbed through the sand/soil profile and stored in the tray or impermeable liner for future use. Excess water can be transmitted away from the system to maintain the optimum distance from “free water”, at the bottom of the system to the surface of the sand/soil profile. This results in the proper air-to-water ratio for plant growth. The spacers allow you to increase the depth of the profile without adding significantly to the weight of the whole system.

The spacer-system will work when using a conventional sprinkler system, a drip-irrigation system, an ECS System or a combination of systems that may be required because of the particular vegetation grown in the different sand/soil profiles.

Geo-foam, available from Styrotech, in Brooklyn Park, Minn., is one of the preferred spacers particularly because it is relatively inert and will not break down. It is used in the road-building industry, is easy to handle and will tend to stay in position once it is placed.

It will be appreciated that the use of soil stabilizers is becoming more common. Sites built on unstable soil can be improved by incorporation of non-degradable soil stabilizing products—generally plastics or poly-olefin fabrics. Products such as Dupont® Typar® Blankets; Netlon® Reflex® Advanced Turfs Mesh Elements; Tensar® meshes; Conwed® oriented plastic netting; Conwedeperforated tubing products; Geo-Grids® polymeric fibers; stabilizing fabrics, stabilizing fibers; Enka Mate products and the like are all products that can stabilize sand and high-organic-matter soils, thereby enhancing the load-bearing characteristics of the sand/soil matrix.

The use of load-bearing enhancers in the upper root-zone of the soil profile, such as Reflex® Mesh Elements, Netting for Geo-Stabilization, Enka Mat®) type products and the like will enhance the load-bearing capacity of the preferred system. Compaction of the sand/soil matrix will be minimized when the sand/soil matrix is exposed to surface traffic and intrusions from lawn chairs, tables, etc. also minimized when such load-bearing enhancers are included. These load-bearing enhancers will provide puncture resistance and reduce compaction, yet not impede the normal water flows or capillary rises of the soil profile. In many instances, these enhancers will increase the drainage properties of the profile.

The above-described features and advantages, along with other various advantages or features of novelty of the present invention are pointed out with particularity in the claims annexed hereto and forming a part hereof. For a better understanding of the invention, its advantages, and objects obtained by its use, however, reference should be made to the drawings which form a further part hereof and to the accompanying description of preferred embodiments of the present invention.

### BRIEF DESCRIPTION OF THE DRAWINGS

Referring now to the drawings, in which like referenced numerals referred to equivalent elements in a series of embodiments of the present invention:

FIG. 1 is an exploded schematic perspective view of a preferred tray system 6 for a modular plant growth system 26 (not shown) that is designed to include a plurality of at least partially contained, reduced weight plant growth mediums (not shown), which can be components of a green space on a roof of a building structure (not shown), a playing field of a spectator pavilion (not shown) or a plant growth area of any architecturally designed building project (not shown) in which a PCRW plant growth medium is desired;

FIG. 2 is a schematic plan view of a single tray structure 20 that retains two air incorporating solid foam waffle members 42 on each side of an ECS chamber 30 within the tray structure 20;

FIG. 3 is a schematic partial cross sectional view of an at least partially contained, reduced weight plant growth medium (PCRW plant growth medium) 2 of the present invention as it would appear from the line 3-3 of FIG. 2 if the tray structure 20 in FIG. 2 included a drainage layer 16 and a sand/soil mixture 14 to make up a sand/soil matrix 12 of the schematic representation shown in FIG. 3 that also shows a sand/soil matrix including polymeric mesh elements 54 in an upper root-zone 37 of the sand/soil matrix 12, as well as grass plants 50 planted in the upper root-zone 37 of the sand/soil matrix 12;

FIG. 4 is a schematic top plan view of an alternate tray structure 20′ including two side by side solid, air incorporating waffle members 42 including openings 44 for containing the sand/soil mixture 14 (not shown) to provide a connection between the upper root-zone and the drainage layer 16 in an, at least partially contained, reduced weight plant growth medium;

FIG. 5 is a schematic cross sectional view of the alternate tray structure 20′ shown in FIG. 4 as seen from the line 5-5 of FIG. 4;

FIG. 6 is a schematic plan view of an alternate tray system 6″ designed to provide a part of the containing elements of an alternate modular plant growth system (not shown) or a preferred green space (not shown) either on a roof of a building structure or as a part of any other structure;

FIG. 7 is a schematic plan view of a limited segment of the alternate tray system 6″ including tray structures 20″ shown in FIG. 5, but also showing parts of one of the PCRW plant growth mediums, before the inclusion of the materials of the drainage layer or the remaining materials in the sand/soil matrix, including a series of ECS chambers 30″ interconnected by fluid transfer pipes 32″, along with a series of solid, air encapsulating foam spacers 40″ provided to reduce the weight of the sand/soil matrix by displacing the sand/soil mixture within the PCRW plant growth medium;

FIG. 8 is a schematic cross sectional view of an alternate green space or modular plant growth system 26′″ on a roof 80 of a building structure 81, which would be generally the same as a modular plant growth system on a floor of a spectator pavilion (not shown), showing parts of a plurality of, at least partially contained, reduced weight growth mediums 2′″ incorporated into the larger green space provided by the modular plant growth system 26′″;

FIG. 9 is a partial schematic plan view of a portion of the alternate tray structure 20 for supporting an alternate reduced weight plant growth medium including a plurality of sand/soil displacement structures 40, namely, enclosed or closed bottles 48;

FIG. 10 is a partial schematic cross sectional view as it would be seen from line 10-10 of FIG. 9, if the sand/soil displacement structures 40 were a series of enclosed air containing compartments 48 of the type shown in FIG. 10, as they would appear when surrounded by the sand/soil mixture 14 and stones 17 in the stone drainage area 16, in a PCRW plant growth medium of the present invention;

FIG. 11A is a partial schematic cross sectional view as it would be seen from line 10-10 of FIG. 9, but if the sand/soil displacement structures 40 were instead concave, air impermeable structures 49 constructed and arranged to capture and retain air when the air impermeable structures 49 are inverted and open to the bottom within a saturation zone 68, but resting on top of the drainage zone 16, as shown in FIG. 10 and as further surrounded by the sand/soil mixture 14 as it would be if it was the PCRW plant growth medium 2″ of the present invention;

FIG. 11 B is a partial schematic cross sectional view as it would be seen from line 10-10 of FIG. 9, but if the sand/soil displacement structures 40 were an air and water permeable sand/soil displacement structure 51 that rest on the bottom 22 of a tray structure 20, rather than on a stone drainage layer 16 in the alternate PCRW plant growth medium 2″;

FIG. 12 is a schematic perspective view of a prior art building structure 86 having a plant growth area 87 on a roof 88;

FIG. 13 is a schematic perspective view of a building structure 90 of the present invention having a roof 91, similar to the building structure 86 shown in FIG. 12, but showing an alternate modular plant growth system 26″″ of the present invention residing upon an upper surface 92 of the roof 91;

FIG. 14 is a schematic perspective view of a spectator pavilion 93 of the present invention including a spectator seating area 94 providing a plurality of seats 95 (shown only in part) in which spectators can be seated, and a playing area 3, including a PCRW plant growth medium 2′″″ having a single tray structure 20′″″ supported by a transport mechanism 96 constructed and arranged to permit the playing area to be moved in and out of the pavilion 93 preferably on a plurality of wheels 97;

FIG. 15 is a schematic plan view of the single tray structure 20, similar to that shown in FIG. 4, except that displacement structures 42, 45 rest on the bottom 22 of the tray structure 20, with the exception of an area cut away from a bottom (not shown) of each of the displacement structures 42, 45 to provide a channel 43 (shown in phantom) that permits water (not shown) to flow from side to side within the tray structure 20 and to drain out of the tray structure 20 through fluid transfer pipes 35;

FIG. 16 is a schematic cross sectional view of the tray structure 20 shown in FIG. 15 as seen through the line 16-16 of FIG. 15;

FIG. 17 is a schematic partial cross sectional view similar to that of FIG. 8, but showing a portion of a modular plant growth system 10 having a plurality of alternate tray structures 20″″″ having a permeable bottom 22″″″ that is permeable to air and water, but prevents sand from leaving the tray structure 22″″″ in order to provide a perched water table system on a roof 80;

FIG. 18 is a schematic partial plan view of an alternate tray system 106 of the present invention illustrating a swing joint 133 that allows the water level to be managed;

FIG. 19 is a partial cross-sectional view of the alternate tray system 106, as seen from line 19-19 of FIG. 18, illustrating the swing joint 133 and the modified single-shell embodiment of an ECS chamber 130; and

FIG. 20 is a partial cross-sectional view of an alternate tray system 206 similar to tray system 106 as tray system 106 would be seen from line 20-20 in FIG. 18, but shown fluid communication between the plurality of tray structures 120 of tray system 206 that rest on a sloped support surface 189.

### DETAILED DESCRIPTION OF THE PREFERRED EMBODIMENTS

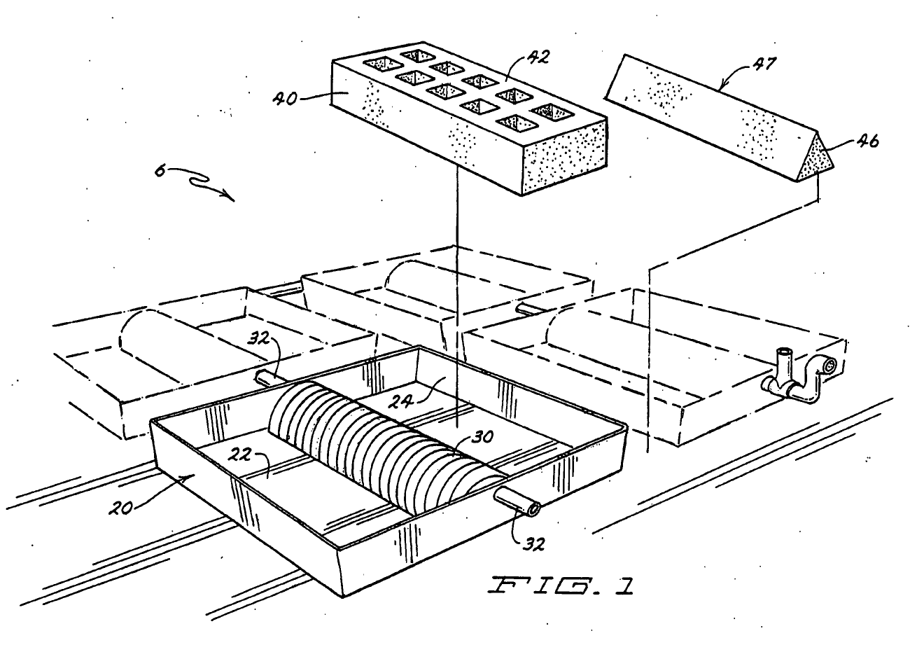

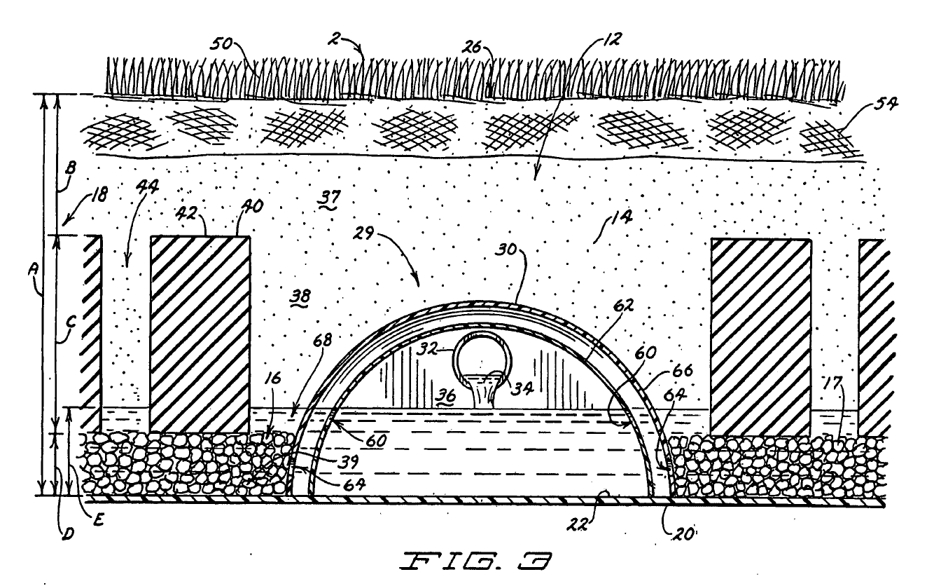

Referring now to the drawings, and specifically to FIGS. 1-3, the elements of a preferred, at least partially contained, reduce weight plant growth medium 2 is shown (see FIG. 3 for partially cross sectional view). The preferred, at least partially contained, reduced weight plant growth medium (PCRW plant growth medium) 2 of the present invention includes a sand/soil matrix 12 having a predetermined depth of a sand/soil mixture 14, preferably resting upon a drainage layer 39, more preferably a stone drainage layer 16 including stones 17, selected to prevent the sand/soil mixture 14 from falling substantially into the drainage area 39 so as to prevent drainage out of the drainage layer 39. The sand/soil matrix 12 will have a predetermined depth A, include a depth B of an upper root-zone 37 and a depth C of a lower root-zone 38; and in preferred embodiments, such as the ECS System shown in FIG. 3, the drainage layer 39 having a predetermined depth D. The depths B and C of the upper root-zone 37 and the lower root-zone 38 are combined to provide a sand/soil depth or profile 18.

The sand/soil matrix will preferably rest in a tray structure 20 having a bottom 22 and four sides 24. The PCRW plant growth medium 2 preferably includes an air incorporating sand/soil displacement structure 40 such as a waffle member 42. The waffle member 42 is made of a rigid, close cell foam material that is preferably generally inert and resists structural change over time. One such foam material is cast geo-foam from STYROTECH, Brooklyn Park, Minn. It will be appreciated that there are numerous other such foams, some of which are discussed below. In addition, however, it will be appreciated that in alternate embodiments, the waffle member 42 could be constructed as an enclosed compartment or a plurality of adjacent compartments that encapsulate air (not shown).

The air incorporating sand/soil displacement structure 40 will preferably rest upon the drainage layer 39, preferably the stone drainage layer 16, on the bottom 22 of the tray structure 20. It will be appreciated that other large particulate objects may be used to form the stone drainage layer 16 in alternate embodiments, and that stones 17 are not a requirement of the present invention, but rather that an average particulate size is preferably at most about eight times larger than the average particulate size of the sand/soil mixture above or the sand/soil mixture will migrate into the drainage layer to diminish the suitability of the water flow in the drainage layer 39.

In preferred embodiments such as that shown in FIGS. 1-3, a tray system 6, including a plurality of tray structure 20 will be provided so that a modular plant growth system 26, which includes a plurality of PCRW plant growth mediums 2 can be provided.

In preferred embodiments, each of the PCRW plant growth mediums 2 will include an Evaporative Control System (ECS) of the kind disclosed in U.S. Pat. No. 5,921,711 to Sipaila for SUB SURFACE FLUID DISTRIBUTION APPARATUS AND METHOD, the disclosure of which is hereby incorporated herein by reference. To provide an Evaporative Control System (ECS), each tray structure 20 is equipped with an ECS chamber 30. The ECS chambers 30 of adjacent tray structures 20 are inner connected with a conduit 32 to allow water to flow from one tray structure 20 to another and to flow in and out of-the respective ECS chambers 30. As shown in FIG. 3, water 34 will flow into a partially enclosed space 36 created by the ECS chamber 30 as it rests upon the bottom 22 of the tray structure 20. As shown in the partial cross sectional view in FIG. 3, the waffle structure 42 rests upon the stone drainage layer 16 on either side of the ECS chamber 30 and forms a part of the sand/soil matrix 12. The sand/soil profile 18 will be a predetermined depth A. This predetermined depth could possibly be as little as 6 inches, however, such a sand/soil profile will not generally provide optimal growth conditions for plants growing in the upper root-zone 37 of the sand/soil profile 18. In preferred embodiments, the predetermined depth A of the sand/soil matrix 12 will be at least about 8 inches, preferably about 10 inches, more preferably about 12 inches and even more preferably about 14 inches or more depending upon the desired plants and the depth of the soil profile required by such plants. For example, a deciduous tree or bush, an evergreen tree or bush, or the like, will often generally require a greater depth of profile in order to maintain optimal root growth depth and also, in certain instances, to suitably anchor the respective plants. In each case, the sand/soil profile 18 includes a predetermined height B for the upper root-zone 37, a predetermined height C for the lower root-zone 38 and a predetermined height D for the drainage layer 39. In alternate embodiments of the present invention, the drainage layer 39 will be about 1 inch, preferably about 2 inches, more preferably about 3 inches, and even more preferably about 4 inches or more; and the lower root-zone will preferably be about 3 inches, more preferably about 4 inches, even more preferably about 4inches, even more preferably about 6 inches, alternately about 7 inches, and alternately as much as 8 or more inches; and the upper root-zone 37 may alternately be at least about 1 inch, preferably at least about 3 inches, more preferably at least about 4 inches and even more preferably at least about 5 inches and alternately at least about 6 inches or more.

In the embodiment shown in FIG. 3, the height of the waffle member 42 is equal to the height of the lower root-zone 38 so that the waffle member 42 will displace the sand/soil mixture 14 in a set of locations in which the waffle member 42 occupies space within the sand/soil matrix 12. Interspersed within the waffle structure 42 are a series of openings 44 that are filled with the sand/soil mixture 14 to interconnect the upper root-zone 37 with the drainage layer 16 proximate these waffle openings 44. In FIG. 3, grass 50 is growing out of the upper root-zone 37 and a plurality of polymeric mesh elements 54 are incorporated into the sand/soil mixture 14 within the soil matrix 12. As will be discussed below, other load-baring enhancers, such as those described above (not shown), may be included in the upper root-zone 37 to both increase the load-barring capacity of the sand/soil matrix, and also to enhance drainage through the upper root-zone in alternate embodiments (not shown).

In FIG. 1, a tray system 6 for a preferred modular plant growth system 26 is shown.

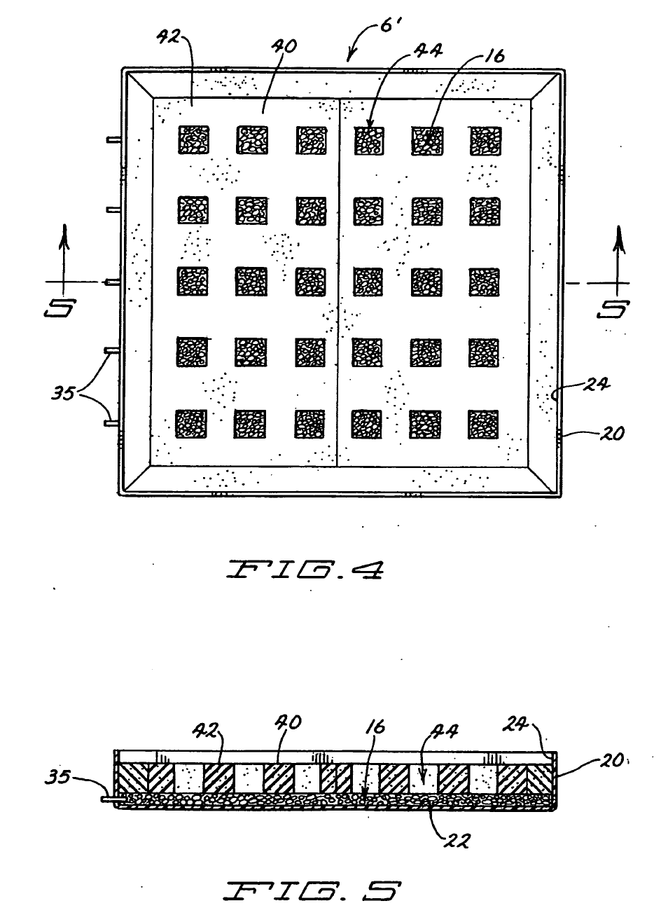

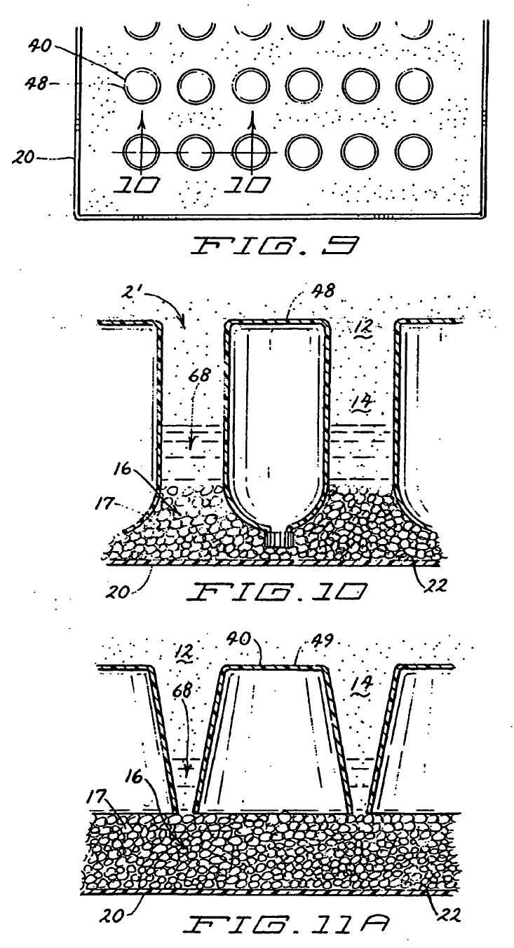

Although alternate PCRW plant growth mediums 2′, 2″ (See FIGS. 9-11), in further alternate embodiments (not shown) having two side by side waffle members 42 as shown in the tray structure 20 shown in FIG. 4, are made without an ECS chamber 30, embodiments including an Evaporative Control System and in ECS chamber 30 are often preferred to address particular needs.

As shown FIG. 3, water 34 can flow into the partially enclosed space 36 defined in part by the ECS chamber 30 and the bottom 22 of the tray structure 20. When the water flows into the partially enclosed space 36 it will flow to its own level, as is in the case in any flow distribution system. In the case of the ECS system 29 shown in FIG. 3, the water will flow out of the partially enclosed space 36 through a series of inner wall apertures 60 passing through an inner wall 62 of the ECS chamber 30 and also out of a series of lower outer wall apertures 64 in the outer wall 66 of the ECS chamber 30. The preferred ECS system is an Evaporative Control System provided by Evaporative Control Systems, Inc., available through the Rehbein Environmental Solutions, Inc., Minneapolis, Minn.

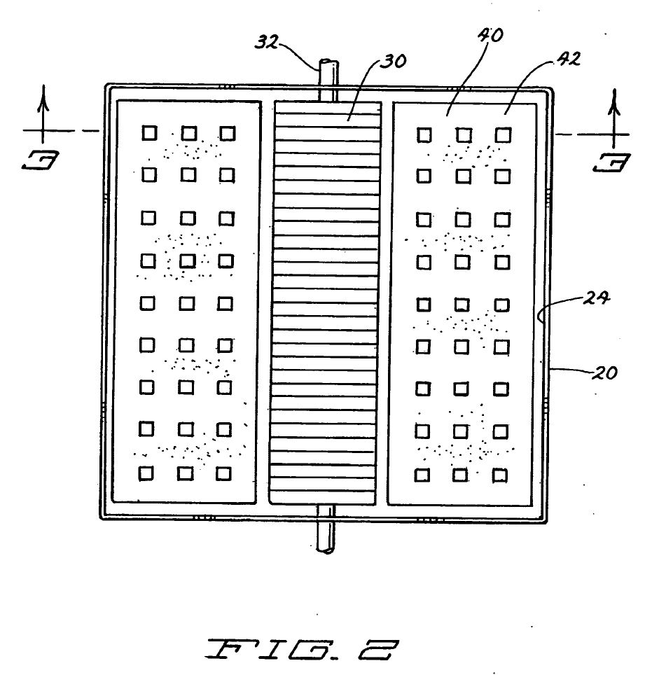

The ECS system 29 includes an ECS chamber 30 made of relatively indestructible polymeric materials. The chamber 30 is designed for non-pressurized water to reach all PCRW plant grown mediums 2 within a modular plant growth system 26 for which the tray system 6, shown in FIG. 1, will be utilized. The preferred conduit 32 is a 2 inch PVC connection/transfer pipe. The preferred tray structure 20 will include an impermeable membrane, having a thickness determined by the puncture potential associated with the envisioned use; preferably a 30 mil. or greater under ground liner to prevent water loss beneath the PCRW plant grown medium 2 and to capture and hold available rain water for future use. The sides 24 (see FIG. 2) of the tray structure 20 preferably create a 3 inch deep saturation zone 68 in each tray structure 20 where the surrounding drainage layer and, preferably a portion of the sand/soil mixture 14 in the lower root-zone 38 of the sand/soil matrix 12. The inner apertures 60 and the outer apertures 64 in the inner and outer walls 62, 66 allow water to transfer from the partially enclosed space 36 within the ECS chamber 30, but to prevent the sand/soil mixture 14 or plant roots (not shown) to enter the chamber 30. In preferred embodiments, the upper root-zone 37 will be preferably at least about 4 inches, more preferably about 5 inches, even more preferably about 6 inches, alternatively about 7 inches, and alternatively as much as 8 inches or more above a saturation zone extending from a saturation zone 68 having a depth E that will preferably extend above the drainage layer 16 at least partially into the lower root-zone 38 as shown in FIG. 3. It will be appreciated, however, that the depth E of the saturated area will depend in large part about the amount of water 34 retained in the tray structure 20 and that it can be much less and even perhaps more than the 3 inches which is generally preferred.

It will be appreciated that Evaporative Control Systems (ECS systems) are well known in the art and are described in the '711 patent to Sipaila that is described above and incorporated herein by reference in the discussion above.

The key aspect of the inclusion of a saturation zone 68 in the PCRW plant growth medium 2 is that the remaining portion of the lower root-zone 38 in locations within the sand/soil matrix 12 in which the sand/soil mixture 14 is not displaced by air incorporating sand/soil displacement structures 40, such as the waffle member 42 shown in FIG. 3, will connect the saturation zone 68 to the upper root-zone 37 and allow water to wick up to the upper root-zone 37 as water forms a constant film around particulate matter within the sand/soil mixture 14 such as grains of sand, with the remaining void space generally occupied by air to supply needed oxygen to the roots that reach into the upper root-zone 37 and also perhaps into the lower root-zone 38. During a growth phase, root hairs (not shown) prefer this environment to absorb water and nutrients from the sand/soil mixture 14. In such a system, a transpiration zone (not shown) will extend from the tip of the deepest plant roots (not shown) in the sand/soil matrix 12 to the top of the highest tip of any growing plant. This is an area of active moisture movement within the plant that not only delivers water and nutrients to all plant tissues during the growth phase, but also regulates plant health. In the ECS system, the plant itself determines its own water needs. The ECS system is generally the one that provides a reliable water reservoir over an extended period of time, as compared to other systems where water may be available in amounts that exceed drain system of the sand/soil matrix or are so limited that insufficient supplies of water are available to wick upwards into the upper root-zone 37 of the sand/soil matrix 14 in such systems. These ECS subsurface irrigation systems are believed to greatly reduce landscaping water expenses, preferably at least in half, reduce fertilization costs, provide for successful plant growth, minimizing the need for replanting; require minimal annual winterization, eliminate annual sprinkler head repair and or replacement, eliminate line breakage repair and replacement; eliminate mineral stained yard ornaments such as fences, bird baths and the like; reduce excess service water that can damage lawn furniture and other items placed on the surface of the PCRW plant growth mediums 2 and virtually eliminate any periods of time when the surface of the green space provided by the PCRW plant growth mediums are unusable due to excess water on or close to the surface.

In preferred tray systems such as the tray system 6 shown in FIG. 1, the respective tray structures 20 are generally positioned relatively adjacent to each other, but, in certain preferred systems, spaced apart to allow the incorporation of an air incorporating sand/soil displacement structure 40 such as the triangular column 47 shown in FIG. 1 that has a triangularly shaped perpendicular cross section as shown on the face 46 of the triangular column 47.

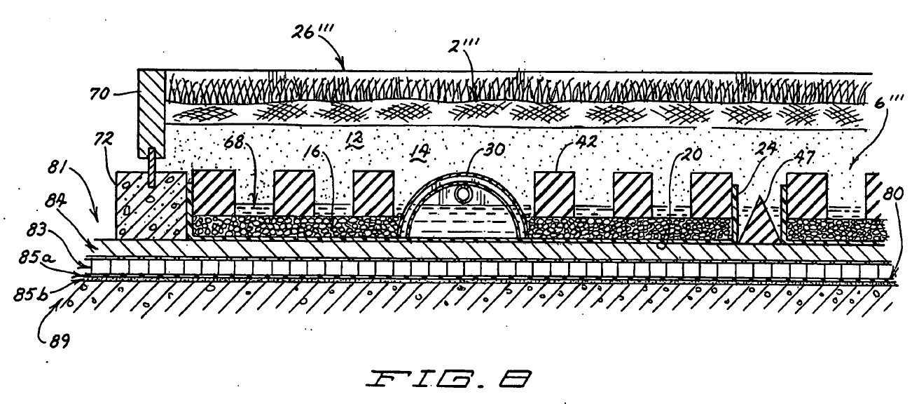

In constructing a modular plant growth system 26 of the present invention, the tray system 6 is put in place with whatever air incorporating sand/soil displacement structures 40, such as the waffle member 42 and the triangular column 47 shown in FIG. 1. Once the air incorporating sand/soil displacement structures 40 are in place and, in preferred embodiments, the ECS chambers 30 in each of the tray structures 20 are in place and inner connected by the drainage conduit 32, stones or other large particulate materials are placed in each of the tray structures 20 on the bottom 22 on each side of the respective ECS chambers 30 to provide a drainage layer 16 having a predetermined height of preferably about 1 to about 2.5 inches or more, so long as the sand/soil mixture 14 in the lower root-zone 38 extends into the saturation zone 68 (also refer to FIG. 3). After the drainage layer 16 is provided, the air incorporating sand/soil displacement structures 40 are preferably placed into the tray structure 20 and the sand/soil mixture 14 is added to a predetermined level that will exceed the height of the sides 24 of the tray structure 20 and spill over into spaces adjacent to the respective tray structures until the sand/soil mixture 14 is limited by a further edging provided around the modular plant growth systems 26 of the present invention such as a structural steel planter edging 70 secured within a concrete or other support 72 as shown in FIG. 8. It is appreciated that in other embodiments, building structures will have other limiting structural components such as a brick wall or the like around the outer edges of a roof, or perhaps treated lumber, concrete retention members or the like that can provide an outer limit to the sand/soil mixture that extends beyond the sides of the respective tray structures and preferred modular plant growth systems 26 of the type shown in FIGS. 3 and 8.

Referring now also to FIGS. 4 and 5, in alternate tray systems 6′ tray structures 20′ can include a variety of air incorporating sand/soil displacement structures 40, such has the waffle members 42 shown in FIG. 4. In these embodiments, there is no ECS system, but water can drain out of a drainage layer 16 through conduits 35 the water is naturally perched when the capillary attraction of the sand is greater than the gravitational forces pulling downward on free water in the tray structure 20′.

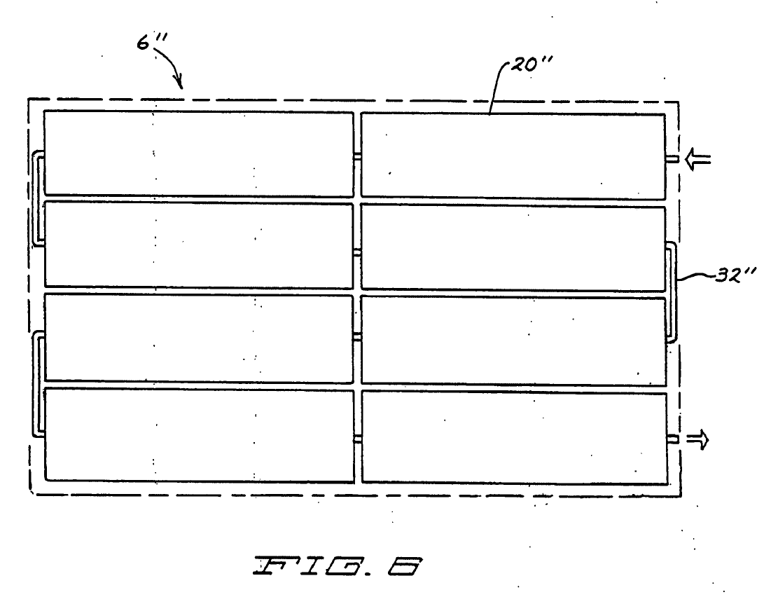

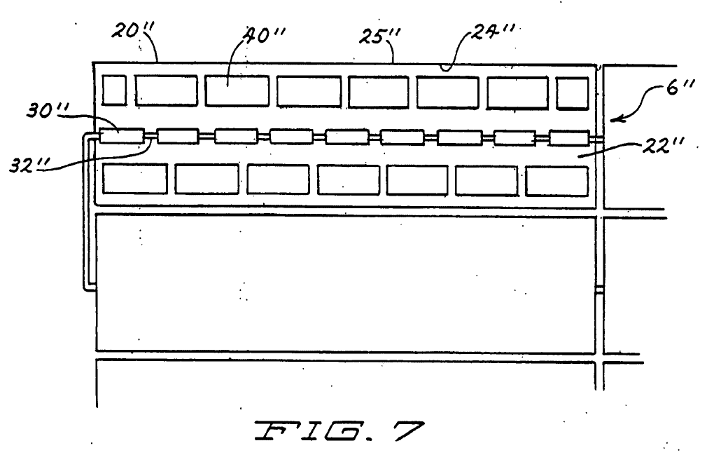

Referring now to FIGS. 6 and 7, an alternate modular plant growth system 26″ is shown schematically in which a series of larger tray structures 20″ each include a plurality of ECS chambers 30″ interconnected in series by connecting conduit 32″ and also include a series of air incorporating sand/soil displacement structures 40′. Each of these larger tray structures 20″ have a bottom 22″ and sides 24″ that enable the tray structure 20″ allow the larger tray structure 20″ to retain water (not shown) can come up as high as an upper edge 25″ of the sides 24″. These larger tray structures 20″ are helpful in building especially large modular plant growth systems 26″ of the type shown schematically in FIGS. 6 and 7. It will be appreciated, however, that the various tray structures 20″, of the present invention may have any shape and that these tray structures 20″ may be arranged in any matter to provide suitable PCRW plant growth mediums for providing green spaces on building structure, roofs, playing fields in spectator pavilions, green spaces in shopping malls, parking garages, highway medians, airport borders and the like.

Referring now also to FIG. 8, the preferred modular plant growth system 26′″ is shown, that is constructed and arranged on a roof 80 of a building structure 81. In this embodiment, the tray system 6′″ preferably rests on an insulation layer 84 made of structural foam material such has geo-foam or the like, which will preferably lie on top of a mat drainage system 83, that in turn preferably rests upon the roof 80 that preferably includes a rubber membrane 85a secured to cement 89 by an adhesive 85b. The mat drainage system 83 will preferably be a system like the HYDRODRAIN and FLORADRAIN products or the like sold by American Hydrotech, Inc., Chicago, Ill. or any similar product designed to provide drainage above a roof or other flat floor structure located below a sand/soil growth medium or the like. The sand/soil mixture in the modular plant growth system 26′″ is limited by the structural steel planter edging 70 that is anchored in a concrete support block 72 that rests upon the structural foam 84. It will be appreciated that the sand/soil mixture 14 will be limited on all sides to provide a suitable green space or modular plant growth system, but that any suitable limiting structure may be used. The Evaporative Control System shown in FIG. 8 is essentially the same as that shown and described in relation to FIG. 3 in the discussion above.

It will be appreciated that the air incorporating sand/soil displacement structures or members 40 of the present invention may include any materials that encapsulate air in any matter and may also include structures that capture air when such structures are concave and are inverted with any opening facing downward and residing in the saturation zone in a parson type PCRW plant growth medium of the present invention. In this regard, it is noted that the air incorporating sand/soil displacement structures may include relatively inert rigid foam material such as expanded polystyrene (EPS) and other synthetic materials such as polyethylene materials, polyisocyanurate materials, polystyrene materials of all kinds, including polystyrols, polyurethane materials and the like that can be used to form rigid close cell foam structures. In this regard, it will be appreciated that any synthetic or other material that is able to provide a rigid air encapsulating solid structure may be used in this regard. For example, air filled pillows capable of supporting the weight of surrounding materials, similar to bubble wrap, may be used. Furthermore, air incorporating sand/soil displacing structures of the present invention will also include closed bottles 48 of the type shown in FIG. 10 and other compartment like structures that enclose and capture air in one or more compartments that can act to displace the sand/soil mixture within the sand/soil matrix of alternate PCRW plant growth mediums of the present invention.

The term “air” is used nonspecifically and is intended to include the gas incorporated into and surrounding the present invention. It will be appreciated that any of the respective sand/soil displacement structures 40 of the present invention may sit directly on the bottom 22 of the respective tray structure 20 without diminishing the effectiveness of the drainage area 16, so long as the fluid within the drainage area can communicate throughout the drainage area. It will also be appreciated that it will be advantageous to either place enough said/soil mixture on top of any of the respective sand/soil displacement structures 40 of the present invention to prevent them from heaving over time during changes in climate or the respective sand/soil displacement structures 40 of the present invention can be secured to the respective tray structures.

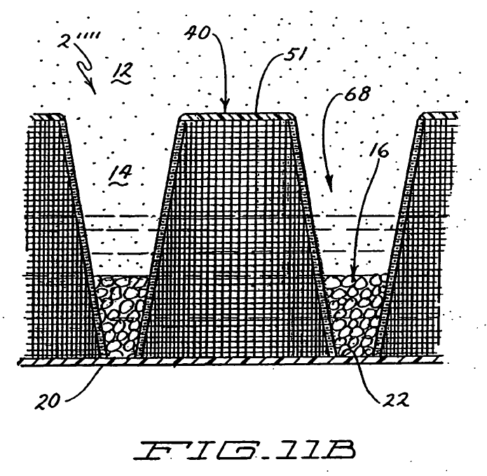

Referring now also to FIGS. 9, 10, 11A and 11 B, in alternate embodiments, the air incorporating sand/soil displacement member or structure 40 can include the closed bottles 48, discussed above and shown in FIG. 10, and air impervious concave structures 49 shown in FIG. 11 A that are inverted so that they are open toward the bottom of the tray 20 and extend into the saturation zone 68 such that an open end of the open structure 49 is below the water level within the tray structure 20 when an appropriate amount of water resides in the tray 20, as shown. Air and water permeable concave structures 51, as shown in FIG. 11 B, can also be used. In each of the alternate embodiments represents schematically in FIGS. 9,10, 11A and 11B, the sand/soil mixture 14 adjacent to the respective air incorporating sand/soil displacement structures 40 (e.g. Bottles 48, concave air impermeable concave structures 49 and air and water permeable concave structures 51) will connect the upper root-zones (not shown), with the saturation zone 68 so that water can wick to the upper root-zone (not shown) and also drain from the upper root-zones (not shown) to the drainage layer 16 when rain or other moisture falls on the top of the upper root-zone (not shown).

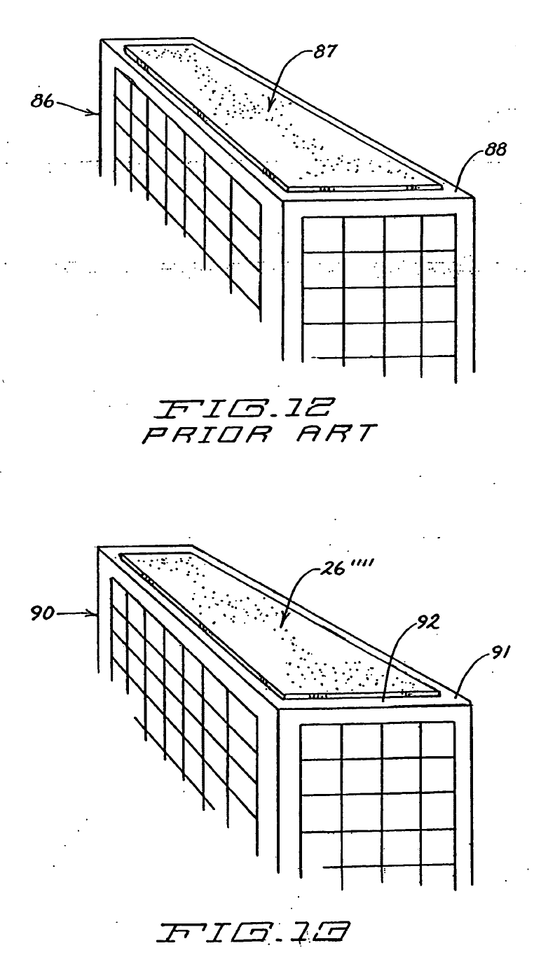

Referring now also to FIG. 12, a prior art building structure 86 is shown having a plant growth area 87 on the roof 88 of the building structure 86. It will be appreciated, that the structural support in the building structure 86 for the roof 88 is significantly greater than would otherwise required for a roof that did not support a plant growth area 87 of the type shown in FIG. 12.

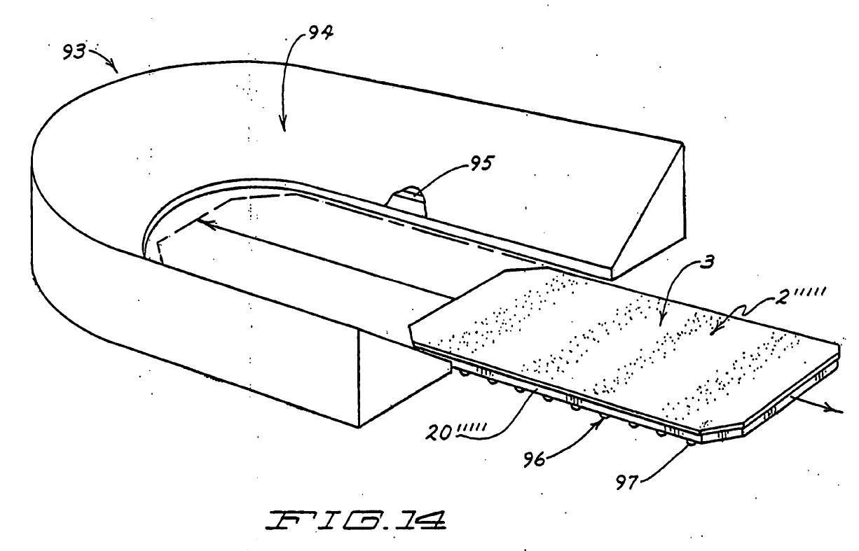

Referring now also to FIG. 13, a building structure 90 having a roof 91 is shown. A modular plant growth system 26″″ is shown residing on the upper surface 92 of the roof 91. The preferred modular plant growth system 26″″ can include any of the respective elements of the respective PCRW plant growth mediums disclosed above and any of the associated elements of the other modular plant growth systems 26, 26′, 26″, 26′″, described herein above that will ensure that the modular plant growth system 26″″ will include reduced weight plant growth mediums of the type described herein above.

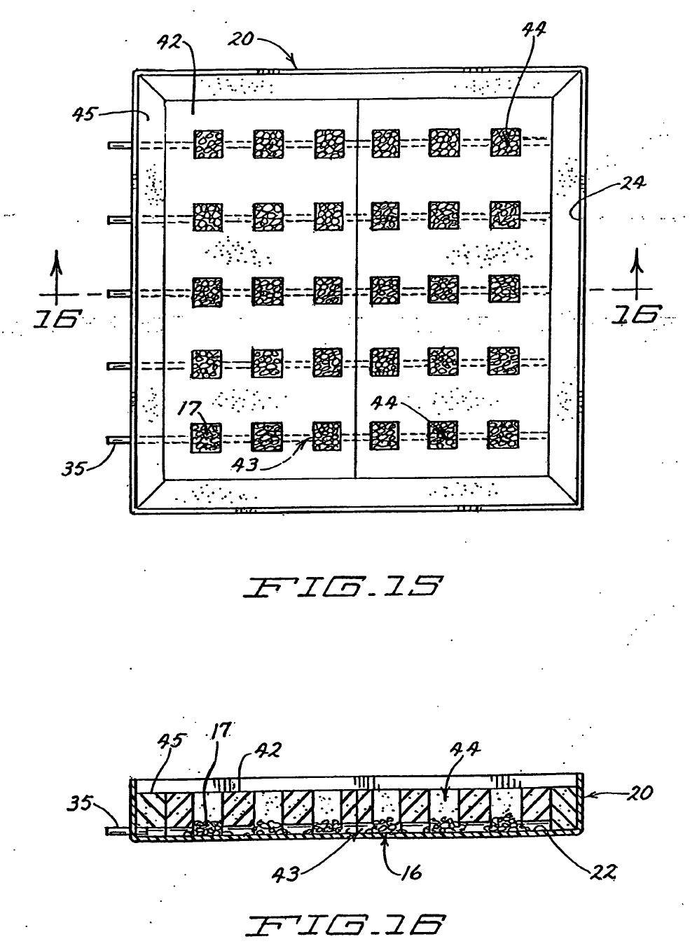

Referring now also to FIG. 14, a spectator pavilion 93 is provided that includes a further embodiment of the present PCRW plant growth medium 2′″″ and a seating area 94 for spectators (not shown). The PCRW plant growth medium 2′″″ includes a single large tray structure 20′″″. In preferred embodiments, like those now under construction near Phoenix, Ariz., for use at the new Cardinal's Stadium, the field area 3, will be a single unit having a transport mechanism 96 including a plurality of wheels 97 that allow the field area 3 to roll in and out of the pavilion 93, so that the pavilion 93 can be used for events that do not require a playing field 3. It is envisioned that other transport mechanisms similar to those known in the art for moving other large sheets may also be used, such as for example a rail system or a pneumatic system employing a fluid to lift the sheet so that it can be easily moved.

In alternate embodiments, a modular plant growth system, having a plurality of PCRW plant growth mediums will be provided as a playing area for various supporting activities in alternate spectator pavilions. In the alternate embodiments, it is envisioned that the modular plant growth system will be supported by a series of tray structures that lie immediately adjacent to one another in a manner similar to the manner in which the playing field at Reliant Stadium in Houston, Tex. is assembled. This allows the alternate modular plant growth system to be disassembled into separate units and transported to an alternate location were the separate units may be watered, fertilized, groomed or the like. Replacement units may then be delivered to the stadium in the manner in which such units are delivered for Reliant Stadium in Houston. It will be appreciated, that the PCRW plant growth medium 2′″″, constructed within a single tray structure 20′″″, and having a plurality of sand/soil displacement structures (not shown) of the type disclosed above, enable the entire enlarged tray 20″″′ to be more easily moved.

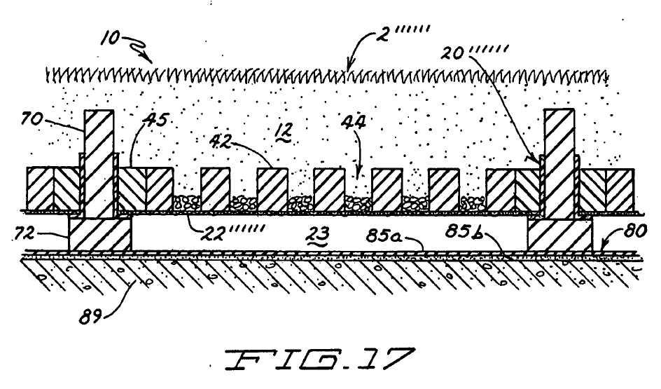

Referring now also to FIGS. 15 and 16, it will be appreciated that tray structure 20 of a natural drainage system may include displacement structures 42 and 45 that rest upon the bottom 22 of the tray structure 20 but provide a channel 43 (shown in phantom in FIG. 15) that will allow water to flow from side to side within the tray 20 and to communicate with the fluid transfer pipes 35. In this way, further weight reduction in a PCRW plant growth medium of the present invention using a tray system 6 similar to that shown in FIGS. 15 and 16. A PCRW plant growth system using such a tray system 6 will have a drainage layer limited to the respective channels 43 and perhaps any fluid communication between the respective channels that can communicate below the respective lower surfaces of the respective displacement structures 42, 45 adjacent to the respective channels. Stones 17 are placed in the drainage layer 16 at the bottom of openings 44 in the waffle member or displacement structure 42, but do not necessarily need to fill the respective channels 43, further reducing the projected weight of a PCRW medium of the present invention utilizing this tray system 6. The only reason stones 17 are required at the bottom of the respective openings 44 is to support any sand/soil mixture 14 that is added to the tray system 6 when completing the building of the PCRW plant growth system of the present invention when utilizing this tray system 6. As previously discussed, the relationship between the respective average sizes of the stones 17 and the anticipated sand/soil mixture particles (not shown) will be arranged to prevent the sand/soil mixture (not shown) from flowing into the drainage layer 16 in a manner such as that set forth in Chapter 9, Installing Subsoil Drains, of Practical Drainage For Golf. Sportsturf And Horticulture, McIntyre and Jakobsen, 2000, Ann Arbor Press, Chelsea, Mich. The stone drainage layer will preferably have particulate materials having a generally uniformed size that is selected to provide an appropriate “bridging capacity” to substantially prevent passage of particles from the sand/soil mixture in the lower root-zone from entering the stone drainage layer as more fully discussed in this Chapter. This entire book, Practical Drainage For Golf. Sportsturf And Horticulture, Mcintyre and Jakobsen, 2000, Ann Arbor Press, Chelsea, Mich., is hereby incorporated herein by reference.

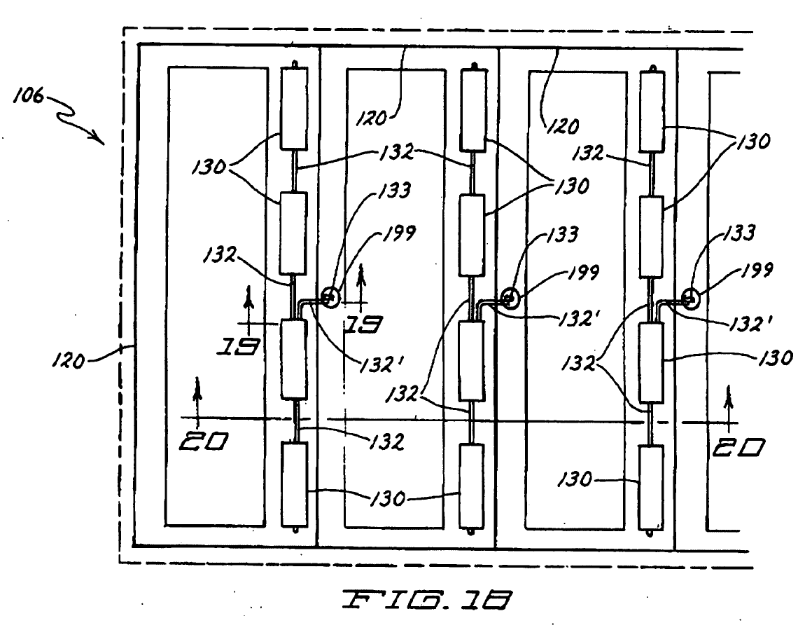

Referring now also to FIG. 17, a further modular plant growth system 10 is shown in a partial schematic cross-sectional view showing a PCRW plant growth medium 2″″″ at least partially contained in an alternate tray structure 20″″″ have a mesh type screen bottom 22″″″ that allows free water (not shown) to drain out of the sand/soil matrix 12 until the remaining moisture in the matrix 14 is retained due to surface tension, capillary forces and other forces that retain water in a sand/soil matrix 14 of the kind envisioned. An open space 23 is maintained below the mesh screen 22″″″, creating a perched water table system similar to that provided in a plurality of modular field unit used to construct the field area at Reliant Stadium in Houston, Tex. The present modular plant growth system 10, is much lighter and easier to move than the modular system at Reliant Stadium, however, because a plurality of sand/soil displacement structures are used to displace the sand/soil mixture 14 in the sand/soil matrix 12 in the manner of the present invention described herein above. For that reason, it can be placed on a roof 80 as shown in FIG. 17.

The present invention also includes methods of building a reduced weight between space and a building structure having a roof, the method comprising the steps of providing and at least partially contained, reduced weight plant growth medium of the present invention; and placing the at least partially contained, reduced weight plant growth medium on the roof of the building structure.

A method of building a reduced weight modular growth system is also provided, the method comprising of steps of providing a plurality of at least partially contained, reduced weight plant growth mediums of the present invention, and placing the plurality of at least partially contained, reduced weight growth mediums adjacent to one another to form the modular plant growth system.

It will be appreciated that the PCRW plant growth mediums of the present invention will be helpful to designers and builders throughout the world in building structures providing green spaces, because the weight to the PCRW plant growth mediums are significantly diminished from those of prior times such as the rooftop green space or plant growth area 87 shown in FIG. 12. In such a green space recently built in Minneapolis, Minn., the weight of the green space was 110 lbs. per square foot. In preferred embodiments of the present invention, green spaces, playing fields, rooftop grassy spaces, rooftop gardens, parking garage plazas and the like, made utilizing a PCRW plant growth medium or a plurality thereof, will have a weight per square foot that will be considerably less than a prior art green space having the same depth of profile, preferably about 10% less, more preferably about 15% less, even more preferably about 20% less, even more preferably about 25% less, even more preferably about 30% less, even more preferably about 35% less, and even more preferably about 40% less or even more, even as much as about 50% less in some alternate embodiments. The key is that, when utilizing the knowledge developed by the present inventors, rooftop and other reduced weight green spaces can be designed to be whatever specifications that a structure can accommodate be simple adding air incorporating sand/soil displacement structure of the present invention to displace the heavier sand/soil mixture to reduce weight, while still providing the necessary connection between the upper root-zone and the drainage layer or the saturation zone where water is generally made available to wick its way to the upper root-zone to provide moisture and nutrients to the plants growing in the upper root-zone.

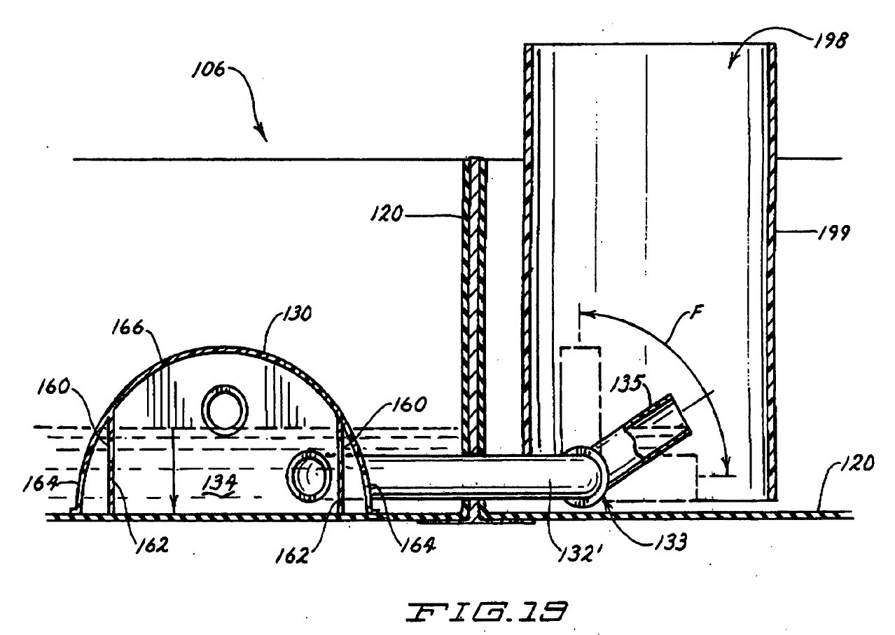

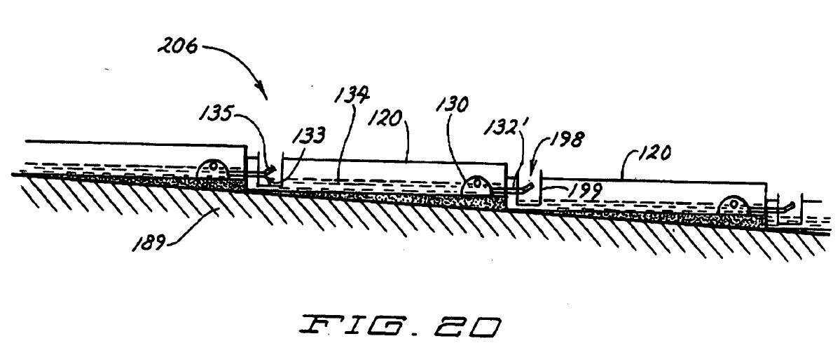

Now also referring to FIGS. 18-20, illustrating alternate embodiments of tray systems 106, 206 for modular plant growth systems (not shown). In the embodiment shown In FIGS. 18 and 19, as with previous embodiments, the tray system 106 includes a plurality of tray structures 120. The tray structures 120 have modified ECS chambers 130, similar to those described above, but have a single shell structure in which the inner wall 62 of the ECS chamber 30, shown in FIG. 3, is functionally replaced by an inner side wall 162 including inner apertures 160. The outer apertures 164 remain in the outer wall 166. The respective ECS chambers 130 in each tray 120 are interconnected by conduits 132 that allow water 134 to flow from one ECS chamber 130 to the next. These further embodiments further include a second conduit 132′ that passes from an ECS chamber 130 in one tray 120 to a swing joint 133 in an adjacent tray 120. The swing joint 133 functions as a manual switch to control the level of water into a void 198 surrounding the swing joint 133, which is defined by a partial enclosure 199, and in the adjacent tray 120. The swing joint 133 has fluid transfer conduit 134 and a pivotally connected end 135 that can be raised or lowered to manage water flow through the end 135 from one tray 120 to another. With particular reference to FIG. 19, the swing joint 133 may be accessed through the void 198. Once accessed, the swing joint 133 may be positioned at a multitude of different angles F, as desired. The higher the end 135 of the swing joint 133 is positioned, the higher the water level in the void 198 or in the ECS chamber 130 in the adjacent tray 120 must be in order for water 134 to pass there between.

It will be appreciated that communication of water 134 from one tray 120 to an adjacent tray 120 can be completely stopped if the end 135 of the swing joint 133 is raised above the water level in either tray 120.

Attention is now additionally directed to FIG. 20 showing a cross-sectional view of an alternate tray system 206 placed on a sloped surface 189. Such a sloped surface may arise in a parking ramp or on certain other surfaces such as a sloped roof or the like. FIG. 20 illustrates the tapered or sloped nature of the structure 189 supporting the alternate tray system 206 for a modular plant growth system (not shown) of the present invention that will have all other features and aspects of the present invention described above. In alternate embodiments, an alternate tray system (not shown) is also contemplated in which each respective tray structure (not shown) will adopt the slant or slope of the surface directly below each tray structure and respective swing joints (not shown) are used to manage fluid transfer between respective adjacent tray structures (not shown) and fluid levels in each of the respective tray structures (not shown).

It is to be understood that, even though numerous characteristics and advantages of various embodiments of the present invention have been set forth in the foregoing description, together with details of the structure and function of various embodiments of the present invention, this disclosure is illustrative only, and changes may be made in detail, especially in matters of shape, size, arrangement of parts, within the broad principals of the present invention, to the full extent indicated by the broad general meaning of the terms in which the appended claims are expressed.

What is claimed is:

1. An at least partially contained, reduced weight plant growth medium comprising:
a sand/soil matrix in which plants can grow; the sand/soil matrix including a sand/soil mixture; the sand/soil mixture having a first weight per unit area;
the sand/soil matrix being constructed and arranged to provide a first uninterrupted sand/soil depth of the sand/soil mixture in a plurality of first zones proximate a first set of isolated locations that is sufficient to support growth of plant species and a second sand/soil depth of the sand/soil mixture in a plurality of second zones proximate a second set of isolated locations that is less than the first uninterrupted sand/soil depth, the sand/soil matrix including at least one sand/soil displacement structure proximate the plurality of second zones, the sand/soil displacement structure having a weight per unit area that is less than the first weight per unit area, such that at least one of the plurality of second zones will have a second weight per unit area that is substantially less than the first weight per unit area.

2. The at least partially contained, reduced weight plant growth medium of claim 1, wherein the sand/soil displacement structure is an air incorporating sand/soil displacement structure and the air incorporating sand/soil displacement structure is selected from the group consisting of (a) an interconnected structure constructed and arranged to (1) incorporate an air containing void into the sand/soil matrix and (2) prevent the sand/soil mixture from filling the air containing void in at least one of the plurality of second zones proximate the second set of isolated locations and (b) an air incorporating sand/soil displacement structure that effectively displaces a portion of the sand/soil mixture in the sand/soil matrix in at least one of the plurality of second zones proximate the second set of isolated locations.

3. The at least partially contained, reduced weight plant growth medium of claim 2, wherein the air incorporating sand/soil displacement structure is a structure selected from the group consisting of an enclosed, air containing compartment, an air encapsulating foam material and a concave, air impermeable structure constructed and arranged to capture and retain air when the air impermeable structure is inverted and open to the bottom.

4. The at least partially contained, reduced weight plant growth medium of claim 1, wherein the sand/soil displacement structure is an air incorporating sand/soil displacement structure and the air incorporating sand/soil displacement structure is a solid structure made of an air encapsulating foam material having a predetermined height, the solid structure including a plurality of openings, each of which run an entire length of the predetermined height such that the sand/soil mixture can occupy the entire predetermined height in each opening, thereby providing an uninterrupted sand/soil depth proximate each of the plurality of openings of at least about 1 inch more than the length of the predetermined height.

5. The at least partially contained, reduced weight plant growth medium of claim 1, wherein the sand/soil matrix includes a plurality of sand/soil displacement structures, wherein each sand/soil displacement structure is an air incorporating sand/soil displacement structure and the air incorporating sand/soil displacement structures effectively displace a portion of the sand/soil mixture proximate the second set of isolated locations.

6. The at least partially contained, reduced weight plant growth medium of claim 5, wherein at least a plurality of the air incorporating sand/soil displacement structures are made of an air encapsulating foam material.

7. The at least partially contained, reduced weight plant growth medium of claim 5, wherein at least a plurality of the air incorporating sand/soil displacement structures are enclosed, air containing compartments.

8. The at least partially contained, reduced weight plant growth medium of claim 5, wherein at least a plurality of the air incorporating sand/soil displacement structures are concave, air impermeable structures constructed and arranged to capture and retain air when the air impermeable structure is inverted and open to the bottom.

9. The at least partially contained, reduced weight plant growth medium of claim 5, wherein at least one of the plurality of air incorporating sand/soil displacement structures is selected from the group consisting of air encapsulating foam material, an enclosed, air containing compartment, a concave, air impermeable structure that is constructed and arranged to capture and retained air when the air impermeable structure is inverted and open to the bottom and an air and water permeable structure that is constructed and arranged to prevent the sand/soil mixture from passing into a cavity beneath the air and water permeable structure.

10. The at least partially contained, reduced weight plant growth medium of claim 5, wherein each of the plurality of air incorporating sand/soil displacement structures are selected from the group consisting of air encapsulating foam material, an enclosed, air containing compartment, a concave, air impermeable structure that is constructed and arranged to capture and retained air when the air impermeable structure is inverted and open to the bottom and an air and water permeable structure that is constructed and arranged to prevent the sand/soil mixture from passing into a cavity beneath the air and water permeable structure.

11. The at least partially contained, reduced weight plant growth medium of claim 1, further comprising a tray structure in which the sand/soil matrix resides; the tray structure having a bottom and four sides such that water can be contained in at least a lower portion of the tray structure.

12. The at least partially contained, reduced weight plant growth medium of claim 11, further comprising a subsurface fluid distribution chamber; the subsurface fluid distribution chamber including an enclosure having a fluid transfer opening, at least a portion of the enclosure being a two-walled structure including: an outer shell having a plurality of apertures formed along a first lateral portion thereof; and an inner shell attached to the outer shell in a spaced apart manner, the inner shell having a plurality of inner shell apertures formed along a first lateral portion thereof, wherein the inner shell apertures define, relative to the enclosure, a subsurface fluid level that is greater than a fluid level defined by the outer shell apertures; and wherein each of the plurality of outer shell apertures is of a size enabling the formation of an infiltrated particle bed adjacent to the outer shell apertures, the infiltrated bed being at a level substantially equal to the fluid level defined by the outer shell apertures.

13. The at least partially contained, reduced weight plant growth medium of claim 1, wherein the sand/soil matrix has a sand/soil profile, the sand/soil profile including an upper root-zone, a lower root-zone and a drainage layer, the upper root-zone including synthetic, polymeric load-bearing enhancers that enhance the load-bearing capacity of the sand/soil matrix.

14. The at least partially contained, reduced weight plant growth medium of claim 1, wherein the sand/soil matrix is constructed and arranged to provide an uninterrupted sand/soil depth of at least about 4 inches of the sand/soil mixture in the plurality of first zones and an uninterrupted sand/soil depth of at least about 1 inch of the sand/soil mixture in the plurality of second zones.

15. A building structure, the building structure comprising;
a structural support system;
a roof connected with and constructed and arranged to be above at least a portion of the structural support system; and
an at least partially contained, reduced weight plant growth medium; the at least partially contained, reduced weight plant growth medium including a sand/soil matrix in which plants can grow; the sand/soil matrix including a sand/soil mixture; the sand/soil mixture having a first weight per unit area;
the sand/soil matrix being constructed and arranged to provide a first uninterrupted sand/soil depth of the sand/soil mixture in a plurality of first zones proximate a first set of isolated locations that is sufficient to support growth of plant species and a second sand/soil depth of the sand/soil mixture in a plurality of second zones proximate a second set of isolated locations that is less than the first uninterrupted sand/soil depth, the sand/soil matrix including at least one sand/soil displacement structure proximate the plurality of second zones, the sand/soil displacement structure having a weight per unit area that is less than the first weight per unit area, such that at least one of the plurality of second zones will have a second weight per unit area that is substantially less than the first weight per unit area.

16. The building structure of claim 15, wherein the sand/soil displacement structure is an air incorporating sand/soil displacement structure and the air incorporating sand/soil displacement structure is selected from the group consisting of (a) an interconnected structure constructed and arranged to (1) incorporate an air containing void into the sand/soil matrix and (2) prevent the sand/soil mixture from filling the air containing void in at least one of the plurality of second zones proximate the second set of isolated locations and (b) an air incorporating sand/soil displacement structure that effectively displaces a portion of the sand/soil mixture in the sand/soil matrix in at least one of the plurality of second zones proximate the second set of isolated locations.

17. The building structure of claim 15, wherein the displacement structure is an air incorporating sand/soil displacement structure and the air incorporating sand/soil displacement structure is a solid structure made of an air encapsulating foam material having a predetermined height, the solid structure including a plurality of openings, each of which run an entire length of the predetermined height such that the sand/soil mixture can occupy the entire predetermined height in each opening, thereby providing an uninterrupted sand/soil depth proximate each of the plurality of openings of at least about 1 inch more than the length of the predetermined height.

18. The building structure of claim 15, wherein the sand/soil matrix includes a plurality of sand/soil displacement structures, wherein each sand/soil displacement structure is an air incorporating sand/soil displacement structure and each of the air incorporating sand/soil displacement structures effectively displace a portion of the sand/soil mixture proximate one of the respective second set of isolated locations.

19. The building structure of claim 18, wherein at least a plurality of the air incorporating sand/soil displacement structures are made of an air encapsulating foam material.

20. The building structure of claim 18, wherein at least a plurality of the air incorporating sand/soil displacement structures are enclosed, air containing compartments.

21. The building structure of claim 18, wherein at least a plurality of the air incorporating sand/soil displacement structures are concave, air impermeable structures constructed and arranged to capture and retain air when the air impermeable structure is inverted and open to the bottom.

22. The building structure of claim 18, wherein at least one of the plurality of air incorporating sand/soil displacement structures is selected from the group consisting of air encapsulating foam material, an enclosed, air containing compartment, a concave, air impermeable structure that is constructed and arranged to capture and retained air when the air impermeable structure is inverted and open to the bottom and an air and water permeable structure that is constructed and arranged to prevent the sand/soil mixture from passing into a cavity beneath the air and water permeable structure.

23. The building structure of claim 18, wherein each of the plurality of air incorporating sand/soil displacement structures are selected from the group consisting of air encapsulating foam material, an enclosed, air containing compartment, a concave, air impermeable structure that is constructed and arranged to capture and retained air when the air impermeable structure is inverted and open to the bottom and an air and water permeable structure that is constructed and arranged to prevent the sand/soil mixture from passing into a cavity beneath the air and water permeable structure.

24. The building structure of claim 15, further comprising a tray structure in which the sand/soil matrix resides; the tray structure having a bottom and four sides such that water can be contained in at least a lower portion of the tray structure.

25. The building structure of claim 24, further comprising a subsurface fluid distribution chamber; the subsurface fluid distribution chamber including an enclosure having a fluid transfer opening, at least a portion of the enclosure being a two-walled structure including: an outer shell having a plurality of apertures formed along a first lateral portion thereof; and an inner shell attached to the outer shell in a spaced apart manner, the inner shell having a plurality of inner shell apertures formed along a first lateral portion thereof, wherein the inner shell apertures define, relative to the enclosure, a subsurface fluid level that is greater than a fluid level defined by the outer shell apertures; and wherein each of the plurality of outer shell apertures is of a size enabling the formation of an infiltrated particle bed adjacent to the outer shell apertures, the infiltrated bed being at a level substantially equal to the fluid level defined by the outer shell apertures.

26. The building structure of claim 15, wherein the sand/soil matrix has a sand/soil profile, the sand/soil profile including an upper root-zone, a lower root-zone and a drainage layer, the upper root-zone including synthetic, polymeric load-bearing enhancers that enhance the load-bearing capacity of the sand/soil matrix.

27. The building structure of claim 15, wherein the sand/soil matrix is constructed and arranged to provide an uninterrupted sand/soil depth of at least about 4 inches of the sand/soil mixture in the plurality of first zones and an uninterrupted sand/soil depth of at least about 1 inch of the sand/soil mixture in the plurality of second zones.

28. The building structure of claim 15 further comprising a plurality of an at least partially contained, reduced weight plant growth mediums.

29. A spectator pavilion, the spectator pavilion comprising:
a spectator seating area including a plurality of seats in which spectators can be seated; and
a playing area including a field area; the field area including a plurality of field units assembled adjacent to one another to form the field area;
each field unit being an at least partially contained, reduced weight plant growth medium; the at least partially contained plant growth medium including a sand/soil matrix in which plants can grow; the sand/soil matrix including a sand/soil mixture; the sand/soil mixture having a first weight per unit area;
the sand/soil matrix being constructed and arranged to provide a first uninterrupted sand/soil depth of the sand/soil mixture in a plurality of first zones proximate a first set of isolated locations that is sufficient to support growth of plant species and a second sand/soil depth of the sand/soil mixture in a plurality of second zones proximate a second set of isolated locations that is less than the first uninterrupted sand/soil depth, the sand/soil matrix including at least one sand/soil displacement structure proximate the plurality of second zones, the sand/soil displacement structure having a weight per unit area that is less than the first weight per unit area, such that at least one of the plurality of second zones will have a second weight per unit area that is substantially less than the first weight per unit area.

30. The spectator pavilion of claim 29, wherein the sand/soil displacement structure is an air incorporating sand/soil displacement structure and the air incorporating sand/soil displacement structure is selected from the group consisting of (a) an interconnected structure constructed and arranged to (1) incorporate an air containing void into the sand/soil matrix and (2) prevent the sand/soil mixture from filling the air containing void in at least one of the plurality of second zones proximate the second set of isolated locations and (b) an air incorporating sand/soil displacement structure that effectively displaces a portion of the sand/soil mixture in the sand/soil matrix in at least one of the plurality of second zones proximate the second set of isolated locations.

31. The spectator pavilion of claim 29, wherein the sand/soil displacement structure is an air incorporating sand/soil displacement structure and the air incorporating sand/soil displacement structure is a solid structure made of an air encapsulating foam material having a predetermined height, the solid structure including a plurality of openings, each of which run an entire length of the predetermined height such that the sand/soil mixture can occupy the entire predetermined height in each opening, thereby providing an uninterrupted sand/soil depth proximate each of the plurality of openings of at least about 1 inch more than the length of the predetermined height.

32. The spectator pavilion of claim 29, wherein the sand/soil matrix includes a plurality of sand/soil displacement structures, wherein each sand/soil displacement structure is an air incorporating sand/soil displacement structure and each of the air incorporating sand/soil displacement structures effectively displace a portion of the sand/soil mixture proximate each of the respective second set of isolated locations.

33. The spectator pavilion of claim 30, wherein each at least partially contained, reduced weight plant growth medium includes a plurality of the air incorporating sand/soil displacement structures made of an air incorporating sand/soil displacement structure selected from the group consisting of an air encapsulating foam material, an enclosed, air containing compartment, a concave, air impermeable structures constructed and arranged to capture and retain air when the air impermeable structure is inverted and open to the bottom and an air and water permeable structure that is constructed and arranged to prevent the sand/soil mixture from passing into a cavity beneath the air and water permeable structure.

34. The spectator pavilion of claim 30, wherein each at least partially contained, reduced weight plant growth medium includes a tray structure in which the sand/soil matrix resides; the tray structure having a bottom and four sides such that water can be contained in at least a lower portion of the tray structure.

35. The spectator pavilion of claim 34, wherein each at least partially contained, reduced weight plant growth medium includes a subsurface fluid distribution chamber; the subsurface fluid distribution chamber including an enclosure having a fluid transfer opening, at least a portion of the enclosure being a two-walled structure including: an outer shell having a plurality of apertures formed along a first lateral portion thereof; and an inner shell attached to the outer shell in a spaced apart manner, the inner shell having a plurality of inner shell apertures formed along a first lateral portion thereof, wherein the inner shell apertures define, relative to the enclosure, a subsurface fluid level that is greater than a fluid level defined by the outer shell apertures; and wherein each of the plurality of outer shell apertures is of a size enabling the formation of an infiltrated particle bed adjacent to the outer shell apertures, the infiltrated bed being at a level substantially equal to the fluid level defined by the outer shell apertures.

36. The spectator pavilion of claim 29, wherein the sand/soil matrix has a sand/soil profile, the sand/soil profile including an upper root-zone, a lower root-zone and a drainage layer, the upper root-zone including synthetic, polymeric load-bearing enhancers that enhance the load-bearing capacity of the sand/soil matrix.

37. A modular plant growth system; the modular plant growth system comprising:
a plurality of at least partially contained, reduced weight plant growth mediums; each of the plurality of at least partially contained, reduced weight plant growth mediums including a sand/soil matrix in which plants can grow; the sand/soil matrix including a sand/soil mixture; the sand/soil mixture having a first weight per unit area;
the sand/soil matrix being constructed and arranged to provide a first uninterrupted sand/soil depth of the sand/soil mixture in a plurality of first zones proximate a first set of isolated locations that is sufficient to support growth of plant species and a second sand/soil depth of the sand/soil mixture in a plurality of second zones proximate a second set of isolated locations that is less than the first uninterrupted sand/soil depth, the sand/soil matrix including at least one sand/soil displacement structure proximate the plurality of second zones, the sand/soil displacement structure having a weight per unit area that is less than the first weight per unit area, such that at least one of the plurality of second zones will have a second weight per unit area that is substantially less than the first weight per unit area.

38. The modular plant growth system of claim 37, wherein the sand/soil displacement structure is an air incorporating sand/soil displacement structure and the air incorporating sand/soil displacement structure is selected from the group consisting of (a) an interconnected structure constructed and arranged to (1) incorporate an air containing void into the sand/soil matrix and (2) prevent the sand/soil mixture from filling the air containing void in at least one of the plurality of second zones proximate the second set of isolated locations and (b) an air incorporating sand/soil displacement structure that effectively displaces a portion of the sand/soil mixture in the sand/soil matrix in at least one of the plurality of second zones proximate the second set of isolated locations.

39. The modular plant growth system of claim 37, wherein the sand/soil displacement structure is an air incorporating sand/soil displacement structure and the air incorporating sand/soil displacement structure is a solid structure made of an air encapsulating foam material having a predetermined height, the solid structure including a plurality of openings, each of which run an entire length of the predetermined height such that the sand/soil mixture can occupy the entire predetermined height in each opening, thereby providing an uninterrupted sand/soil depth proximate each of the plurality of openings of at least about 1 inch more than the length of the predetermined height.

40. The modular plant growth system of claim 37, wherein the sand/soil matrix includes a plurality of sand/soil displacement structures, wherein each sand/soil displacement structure is an air incorporating sand/soil displacement structure and each of the air incorporating sand/soil displacement structures effectively displace a portion of the sand/soil mixture proximate each of the respective second set of isolated locations.

41. The modular plant growth system of claim 38, wherein each at least partially contained, reduced weight plant growth medium includes a plurality of the air incorporating sand/soil displacement structures made of an air incorporating sand/soil displacement structure selected from the group consisting of an air encapsulating foam material, an enclosed, air containing compartment, a concave, air impermeable structures constructed and arranged to capture and retain air when the air impermeable structure is inverted and open to the bottom and an air and water permeable structure that is constructed and arranged to prevent the sand/soil mixture from passing into a cavity beneath the air and water permeable structure.

42. The modular plant growth system of claim 38, wherein each at least partially contained, reduced weight plant growth medium includes a tray structure in which the sand/soil matrix resides; the tray structure having a bottom and four sides such that water can be contained in at least a lower portion of the tray structure.

43. The modular plant growth system of claim 42, wherein each at least partially contained, reduced weight plant growth medium includes a subsurface fluid distribution chamber; the subsurface fluid distribution chamber including an enclosure having a fluid transfer opening, at least a portion of the enclosure being a two-walled structure including: an outer shell having a plurality of apertures formed along a first lateral portion thereof; and an inner shell attached to the outer shell in a spaced apart manner, the inner shell having a plurality of inner shell apertures formed along a first lateral portion thereof, wherein the inner shell apertures define, relative to the enclosure, a subsurface fluid level that is greater than a fluid level defined by the outer shell apertures; and wherein each of the plurality of outer shell apertures is of a size enabling the formation of an infiltrated particle bed adjacent to the outer shell apertures, the infiltrated bed being at a level substantially equal to the fluid level defined by the outer shell apertures.

44. The modular plant growth system of claim 37, wherein the sand/soil matrix has a sand/soil profile, the sand/soil profile including an upper root-zone, a lower root-zone and a drainage layer, the upper root-zone including synthetic, polymeric load-bearing enhancers that enhance the load-bearing capacity of the sand/soil matrix.

45. A method of building a reduced weight green space on a building structure having a roof; the method comprising the steps of:
constructing an at least partially contained, reduced weight plant growth medium on the roof of the building structure; the at least partially contained, reduced weight plant growth medium including: a sand/soil matrix in which plants can grow; the sand/soil matrix including a sand/soil mixture; the sand/soil mixture having a first weight per unit area; the sand/soil matrix being constructed and arranged to provide a first uninterrupted sand/soil depth of the sand/soil mixture in a plurality of first zones proximate a first set of isolated locations that is sufficient to support growth of plant species and a second sand/soil depth of the sand/soil mixture in a plurality of second zones proximate a second set of isolated locations that is less than the first uninterrupted sand/soil depth, the sand/soil matrix including at least one sand/soil displacement structure proximate the plurality of second zones, the sand/soil displacement structure having a weight per unit area that is less than the first weight per unit area, such that at least one of the plurality of second zones will have a second weight per unit area that is substantially less than the first weight per unit area.

46. A method of building a reduced weight modular plant growth system; the method comprising the steps of: providing a plurality of at least partially contained, reduced weight plant growth mediums; each of the plurality of at least partially contained, reduced weight plant growth mediums including a sand/soil matrix in which plants can grow; each sand/soil matrix including a sand/soil mixture; the sand/soil mixture having a first weight per unit area; the sand/soil matrix being constructed and arranged to provide a first uninterrupted sand/soil depth of the sand/soil mixture in a plurality of first zones proximate a first set of isolated locations that is sufficient to support growth of plant species and a second sand/soil depth of the sand/soil mixture in a plurality of second zones proximate a second set of isolated locations that is less than the first uninterrupted sand/soil depth, the sand/soil matrix including at least one sand/soil displacement structure proximate the plurality of second zones, the sand/soil displacement structure having a weight per unit area that is less than the first weight per unit area, such that at least one of the plurality of second zones will have a second weight per unit area that is substantially less than the first weight per unit area; and
placing the plurality of at least partially contained, reduced weight plant growth mediums adjacent to one another to form the modular plant growth system.

47. The method of claim 46, wherein each of the plurality of at least partially contained, reduced weight plant growth mediums include a tray structure; each tray structure including a fluid conduit through which fluid can flow to or from an adjacent tray structure; the fluid conduit including a swing joint having a pivotal end constructed and arranged to permit the flow of water between such adjacent tray structures to be managed by pivoting the pivotal end upward of downward.

48. A spectator pavilion, the spectator pavilion comprising:
a spectator seating area including a plurality of seats in which spectators can be seated; and
a field area including a field area; the field area including a field unit; the field unit being an at least partially contained, reduced weight plant growth medium residing upon the roof; the at least partially contained plant growth medium including a sand/soil matrix in which plants can grow; the sand/soil matrix including a sand/soil mixture; the sand/soil mixture having a first weight per unit area;
the sand/soil matrix being constructed and arranged to provide a first uninterrupted sand/soil depth of the sand/soil mixture in a plurality of first zones proximate a first set of isolated locations that is sufficient to support growth of plant species and a second sand/soil depth of the sand/soil mixture in a plurality of second zones proximate a second set of isolated locations that is less than the first uninterrupted sand/soil depth, the sand/soil matrix including at least one sand/soil displacement structure proximate the plurality of second zones, the sand/soil displacement structure having a weight per unit area that is less than the first weight per unit area, such that at least one of the plurality of second zones will have a second weight per unit area that is substantially less than the first weight per unit area.

49. The spectator pavilion of claim 48, wherein the field area includes a transport mechanism constructed and arranged to support the field unit, the transport mechanism permitting the field unit to move while being supported by the transport mechanism.

50. The spectator pavilion of claim 49, wherein the transport mechanism includes a plurality of wheels permitting the field unit to roll from one position to another while being supported by the transport mechanism.
